SEQUENCE LISTING (11 sequences)

SEQ ID NO: 1 - DNA, 1131 bp, Corynebacterium glutamicum
  ref <308>: DATABASE ACCESSION NUMBER: GenBank/NCgl2640
  ref <309>: DATABASE ENTRY DATE: 2002-03-20
  ref <313>: RELEVANT RESIDUES: (1)..(1131)
atgggcattgagtttaagcgttcaccgcgacccaccctgggcgttgagtgggaaattgcacttgttgatccagaaacacgtgatctagccccgcgcgctgcagaaatactagagattgtggccaagaaccaccctgaggtgcacctcgagcgcgaattcctccaaaacaccgtggagcttgtcaccggagtgtgcgacaccgtccccgaagcggtggcagagctttcccacgatctagatgcgctgaaagaagcagcggattctctcgggcttcggttgtggacctctggatcccacccattttcggatttccgcgaaaacccagtatctgaaaaaggctcctacgacgagatcatcgcgcgcacccaatactggggaaaccagatgttgatttggggcattcacgtccacgtgggcatcagccatgaagatcgcgtgtggccgatcatcaatgcgctgctgacaaattacccacatctgttggcactttctgcaagctctccagcatgggacggacttgataccggttatgcctccaaccggacgatgctctaccaacagctgcctacagccggactgccataccaattccaaagctgggatgaatggtgcagctacatggcggatcaagataaatccggtgtcatcaaccacaccggatccatgcactttgatatccgccccgcatccaaatggggaaccatcgaagtccgcgtggccgattctacctccaacctgcgggaactgtctgccatcgtggcgttgacccactgtctcgtggtgcactacgaccgcatgatcgacgctggcgaagagcttccctccctgcaacaatggcacgtttcggaaaataaatggcgcgcggctaggtatggtctggatgccgaaatcatcatttccagagacaccgatgaagcgatggttcaagacgaactccgccgactagtagcgcaattgatgcctctagccaacgaactcggctgcgctcgtgagcttgaacttgtgttggaaatcctggaacgtggtggtggatacgaacgccaacgcagagtgtttaaagaaactggcagttggaaagctgcagttgatttagcctgcgacgaactcaacgacctcaaagcactggactaa

SEQ ID NO: 2 - protein, 376 aa, Corynebacterium glutamicum
  ref <308>: DATABASE ACCESSION NUMBER: GenBank/NP_601931
  ref <309>: DATABASE ENTRY DATE: 2002-03-20
  ref <313>: RELEVANT RESIDUES: (1)..(376)
MGIEFKRSPRPTLGVEWEIALVDPETRDLAPRAAEILEIVAKNHPEVHLEREFLQNTVELVTGVCDTVPEAVAELSHDLDALKEAADSLGLRLWTSGSHPFSDFRENPVSEKGSYDEIIARTQYWGNQMLIWGIHVHVGISHEDRVWPIINALLTNYPHLLALSASSPAWDGLDTGYASNRTMLYQQLPTAGLPYQFQSWDEWCSYMADQDKSGVINHTGSMHFDIRPASKWGTIEVRVADSTSNLRELSAIVALTHCLVVHYDRMIDAGEELPSLQQWHVSENKWRAARYGLDAEIIISRDTDEAMVQDELRRLVAQLMPLANELGCARELELVLEILERGGGYERQRRVFKETGSWKAAVDLACDELNDLKALD

SEQ ID NO: 3 - DNA, 642 bp, Corynebacterium glutamicum
  ref <308>: DATABASE ACCESSION NUMBER: GenBank/AY217661
  ref <309>: DATABASE ENTRY DATE: 2003-07-30
  ref <313>: RELEVANT RESIDUES: (1)..(642)
gtggctgctagcgcttcaggcaagagtaaaacaagtgccggggcaaaccgtcgtcgcaatcgaccaagcccccgacagcgtctcctcgatagcgcaaccaaccttttcaccacagaaggtattcgcgtcatcggtattgatcgtatcctccgtgaagctgacgtggcgaaggcgagcctctattcccttttcggatcgaaggacgccttggttattgcatacctggagaacctcgatcagctgtggcgtgaagcgtggcgtgagcgcaccgtcggtatgaaggatccggaagataaaatcatcgcgttctttgatcagtgcattgaggaagaaccagaaaaagatttccgcggctcgcactttcagaatgcggctagtgagtaccctcgccccgaaactgatagcgaaaagggcattgttgcagcagtgttagagcaccgcgagtggtgtcataagactctgactgatttgctcactgagaagaacggctacccaggcaccacccaggcgaatcagctgttggtgttccttgatggtggacttgctggatctcgattggtccacaacatcagtcctcttgagacggctcgcgatttggctcggcagttgttgtcggctccacctgcggactactcaatttag

SEQ ID NO: 4 - protein, 213 aa, Corynebacterium glutamicum
  ref <308>: DATABASE ACCESSION NUMBER: GenBank/NP_602128
  ref <309>: DATABASE ENTRY DATE: 2002-03-20
  ref <313>: RELEVANT RESIDUES: (1)..(213)
MAASASGKSKTSAGANRRRNRPSPRQRLLDSATNLFTTEGIRVIGIDRILREADVAKASLYSLFGSKDALVIAYLENLDQLWREAWRERTVGMKDPEDKIIAFFDQCIEEEPEKDFRGSHFQNAASEYPRPETDSEKGIVAAVLEHREWCHKTLTDLLTEKNGYPGTTQANQLLVFLDGGLAGSRLVHNISPLETARDLARQLLSAPPADYSI

SEQ ID NO: 5 - DNA, 35 bp, Artificial
  Primer 2640-fwd-BglII
ccgctgctgctggtggcgctagatctgctaacggc

SEQ ID NO: 6 - DNA, 35 bp, Artificial
  Primer 2640-rev-BglII
atgtgttgggagatctcttaagttatttagtccag

SEQ ID NO: 7 - DNA, 23 bp, Artificial
  oligonucleotide Tn5531-Eco
cgggtctacaccgctagcccagg

SEQ ID NO: 8 - DNA, 29 bp, Artificial
  Primer 2640-SacB1
gagagggcccatcagcagaacctggaacc

SEQ ID NO: 9 - DNA, 19 bp, Artificial
  Primer 2640-SacB2
gatccagaggtccacaacc

SEQ ID NO: 10 - DNA, 20 bp, Artificial
  Primer 2640-SacB3
gatggttcaagacgaactcc

SEQ ID NO: 11 - DNA, 30 bp, Artificial
  Primer 2640-SacB4
gagagtcgaccagaatcaattccagccttc

(19) United States
(12) Patent Application Publication
(10) Pub. No.: US 2009/0053778 A1
(43) Pub. Date: Feb. 26, 2009

(54) Microorganisms for producing sulfur-containing compounds

(75) Inventors: Uwe Sauer, Zurich (CH); Jorg Mampel, Bensheim (DE); Oskar Zelder, Speyer (DE); Andrea Herold, Ketsch (DE); Corinna Klopprogge, Mannheim (DE); Hartwig Schroder, Nussloch (DE); Stefan Haefner, Speyer (DE)

Correspondence Address: LAHIVE & COCKFIELD, LLP;FLOOR 30, SUITE 3000, ONE POST OFFICE SQUARE, BOSTON, MA, 02109, US

(73) Assignee: FRANCE TELECOM, PARIS (FR)

(21) Appl. No.: 11/632,693

(22) PCT Filed: Jul. 20, 2005
(86) PCT No.: PCT/EP05/07925

§ 371 Date: Oct. 27, 2008

(30) Foreign Application Priority Data
Jul. 20, 2004 (DE) 10-2004-035-052.3

(51) Int. Cl. C12P 13/12; C12N 1/21; C12P 13/04; C12P 17/18; C12P 17/16; C12P 7/64

(52) U.S. Cl. 435/113; 435/252.31; 435/252.33; 435/252.35; 435/252.34; 435/106; 435/119; 435/118; 435/134

(57) ABSTRACT

The present invention relates to microorganisms and methods for producing at least one sulfur-containing compound.

The present invention relates to microorganisms and methods for producing at least one sulfur-containing compound. In particular, the present invention relates to methods for producing L-methionine or L-cysteine in Corynebacterium glutamicum.

Amino acids are used as additives for different purposes in the nutrition of animals and humans. For instance, glutamate is typically used as flavor enhancer. Methionine is an essential amino acid which has to be taken in with food. Both methionine and other sulfur-containing amino acids like cysteine are essential not only for protein biosynthesis, but also serve as precursors for different metabolites like glutathione, S-adenosylmethionine and biotin and furthermore as methyl group donors in different cellular processes.

The worldwide consumption of amino acids is currently estimated to be higher than two million tons. The annual need for amino acids like glutamate or fodder additives mainly consisting of L-lysine, L-methionine, and L-threonine is estimated to be higher than one million ton per amino acid. The annual need for amino acids used in pharmaceutical products alone amounts to 15,000 tons (Kusumoto, I. (2001), J. Nutr., 131, 2552-2555).

While at the beginning of the last century amino acids were mainly produced chemically or by means of extraction, the production of specific amino acids like, for example, glutamate is nowadays mainly performed by fermentation processes. Herein, specific microorganisms like, for example, Escherichia coli and Corynebacterium glutamicum have proven to be particularly suitable for the fermentative production of amino acids. The production of amino acids by fermentation has, in particular, the advantage that the naturally usable L-amino acids are produced exclusively, whereas from chemical syntheses usually a racemic mixture is formed, which often requires time- and cost-intensive processing.

In particular, there is a need for so-called sulfur-containing compounds like methionine, homocysteine, S-adenosylmethionine, glutathione, cysteine, biotin, thiamine, lipoic acid etc., which are used in many industrial branches including food, fodder, cosmetics, and pharmaceutical industry. These substances, which are also referred to as sulfur-containing fine chemicals, comprise organic acids, proteinogenic and non-proteinogenic amino acids, vitamins, and cofactors.

It was attempted to use microorganisms like E. coli and C. glutamicum for producing the previously mentioned sulfur-containing compounds. Due to the great importance of the sulfur-containing compounds, in particular attempts at improving methods for the production of said microorganisms were made, for example by technical fermentation measures like stirring and oxygen supply, composition of the nutritive media, etc.

Beside this, it was attempted, by classical strain selection, to develop mutant strains of the mentioned microorganisms, which produce the mentioned sulfur-containing compounds at a considerably larger scale than the respective non-mutated wild-type.

Herein, the use of microorganisms like E. coli or C. glutamicum has turned out to be difficult, in particular in fermentative industrial large-scale production of sulfur-containing amino acids like methionine and cysteine. There is, however, a strong need for exactly those amino acids, as they are, inter alia, precursor compounds for several more of the previously mentioned sulfur-containing compounds.

Most probably, the biosynthetic or metabolic pathways leading to the production of methionine and cysteine are the reason that industrial-scale fermentative production of methionine and cysteine in microorganisms has up to now not turned out to be of economical interest. Biosynthesis of cysteine and methionine in E. coli is generally known (see for example Voet and Voet (1995) Biochemistry, John Wiley and Sons, Inc. USA). Biosynthesis of methionine and cysteine in Corynebacterium glutamicum is the object of intensive research and has been most currently described in Rückert et al. (Rückert et al. (2003), J. of Biotechnology, 104, 213-228) as well as in Lee et al. (Lee et al. (2003), Appl. Microbiol. Biotechnol., 62, 459-467).

The key step in the biosynthesis of methionine is the incorporation of sulfur into the carbon scaffold. Regularly, the sulfur source is sulfate, which has to be taken up, activated, and reduced by the microorganisms. Said steps lead to a consumption of 7 Mol ATP and 8 Mol NADPH for each Mol of methionine (Neidhardt et al. (1990) Physiology of the bacterial cell: a molecular approach, Sunderland, Mass., USA, Sinauer Associates, Inc.). Thus, methionine is the one amino acid, whose production requires the largest amount of energy from the cell.

Correspondingly, the microorganisms producing methionine have developed metabolic pathways which are under a strict regulation control with respect to the production of methionine, but also of cysteine. This means that, for example, by feedback regulation mechanisms, the activity of metabolic pathways employing methionine is down-regulated as soon as the cell has produced a sufficient amount of methionine.

This has led to the fact that up to now methionine was the only amino acid to be produced exclusively chemically on an industrial scale, although this requires subsequent enantiomeric separation of the L- and D-forms of methionine.

Thus, little is known about methionine-producing variants of Corynebacterium glutamicum. Such mutants were identified as early as 1975 (Kase et al. (1975) Agric. Biol. Chem., 39, 153-160). However, the strains described therein are not of interest for the industrial production of methionine in microorganisms. Since that time, no improvements have been achieved.

The prior art approaches to produce microorganisms allowing an increased production of methionine have up to now been concentrated on identifying those genes which are involved in the biosynthetic pathway of methionine and other sulfur-containing compounds and subsequently increasing the production of methionine or of the other compounds by overexpression or repression of the genes depending on the respective function.

Such attempts are, for example, described in WO 02/10209. Herein, it has turned out to be problematic that said document merely describes the overexpression or repression of individual genes for increasing the production of, for example, methionine. As, in this way, normally only the control mechanism for one synthesis step of the biosynthesis of, for example, methionine is deactivated while other regulatory mechanisms remain unaffected, an increased production of sulfur-containing compounds can thus only be achieved in a limited manner (see also FIG. 3 in Lee et al. vide supra). By overexpression of an individual gene, which is involved in the biosynthesis of sulfur-containing compounds, normally the previously mentioned regulatory mechanisms are thus only uncoupled insufficiently. The increases in the content of methionine or other sulfur-containing compounds, though achieved, but yet still being modest, are not sufficient to render the method worthwhile for fermentative production in microorganisms in an industrial scale.

In the production of sulfur-containing compounds such as methionine in microorganisms, the particular problem arises that the genes of the biosynthetic pathway of methionine can be distributed over the circular genome of microorganisms (see Rückert et al., vide supra). Examples for microorganisms having genes of the biosynthetic pathway of methionine distributed over their genome in this manner are Corynebacterium glutamicum, Escherichia coli, Bacillus like Bacillus subtilis, Serratia like Serratia marcescens, and Salmonella. The distribution of the genes responsible for methionine biosynthesis can be regarded as an implication that those genes are, for example, not organized within an operon, i.e. within a common regulatory unit. If the latter were the case, several regulatory mechanisms could be uncoupled simultaneously and thus a more efficient enhancement of the amount of methionine produced by microorganisms could be achieved for example by influencing, i.e. overexpressing or repressing, the molecular switch, which regulates said operon. The fact that such operons can, in principle, exist despite the distribution of the genes involved in the biosynthesis of methionine in the genome of Corynebacterium glutamicum has recently been shown (Rey et al. (2003), J. Biotechnol., 103, 51-65).

Rey et al. could show that the protein McbR is a transcriptional repressor, which regulates the expression of the following genes which are involved in methionine biosynthesis: metY (coding for O-acetyl-L-homoserine sulfhydrylase), metK (coding for S-adenosyl-methionine synthetase), hom (coding for homoserine dehydrogenase), cysK (coding for L-cysteine synthase), cysI (coding for NADPH-dependent sulfide reductase), and ssuD (coding for alkanesulfonate monooxygenase). All of the previously mentioned enzymes are involved in the methionine and/or cysteine biosynthesis and the authors could correspondingly show that, in a knockout strain of C. glutamicum, increased amounts of methionine are produced for McbR.

In the light of these results there is a strong interest in identifying further factors involved in the central regulation of the biosynthetic pathways for sulfur-containing compounds in microorganisms, as, by overexpressing or repressing (according to function) such central regulatory switches, it should be possible to produce microorganisms producing significantly increased amounts of sulfur-containing compounds.

It is thus a problem underlying the present invention to provide microorganisms, in which it is possible to produce sulfur-containing compounds.

In particular, it is also a problem of the present invention to provide microorganisms and methods for producing sulfur-containing compounds like methionine or cysteine in microorganisms like E. coli or Corynebacterium glutamicum, wherein obtaining significantly increased amounts of the previously mentioned sulfur-containing compounds is made possible by deactivating a central regulatory element of the biosynthetic pathways of the sulfur-containing compounds.

Moreover, it is a problem of the present invention to identify nucleic acid sequences which can be used to produce mutated organisms producing increased amounts of sulfur-containing compounds, like for example methionine and/or cysteine, as compared to their respective wild-type.

These and further problems underlying the present invention, as obvious from the description, are solved by the independent claims.

Preferred embodiments of the present invention are described in the subclaims.

Within the scope of the present invention it was possible, by a novel selection and mutation mechanism in C. glutamicum, to identify a factor, namely NCgl2640 (GenBank accession number of the Entrez database, http://www.ncbi.nlm.nih.gov, SEQ ID NO: 1), which represents a central regulatory switch in the regulation of sulfur-containing compounds in C. glutamicum and, in particular, of methionine. As a homology search with the amino acid sequence encoded by NCgl2640 (GenBank accession number: NP_{—}601931, SEQ ID NO: 2) by means of the BLAST program at the NCBI (http:/www.ncbi.nlm.nih.gov) has led to the result that several homologs exist in other different organisms, like for example in E. coli, it can be assumed that, in light of the conservation of biosynthetic pathways for amino acids and sulfur-containing compounds, in particular between the different microorganisms, said factor is also involved in the central regulation of the biosynthesis pathways of sulfur-containing compounds in the other microorganisms.

As is shown within the scope of the present invention, microorganisms, in which the content and/or the activity of nucleic acids, which are identical or functionally homologous to nucleic acids having the sequence of SEQ ID NO: 1, is reduced as compared to the wild-type, can be used for producing sulfur-containing compounds.

Thus, an object of the present invention relates to a microorganism, in which the content and/or the activity of proteins encoded by nucleic acids that are identical or functionally homologous to nucleic acids having the sequence of SEQ ID NO: 1 is reduced as compared to the wild-type of the microorganism. In the following, this object of the present invention is also described in that the content and/or the activity of nucleic acids that are identical or functionally homologous to nucleic acids having the sequence of SEQ ID NO: 1 is reduced as compared to the wild-type of the microorganism.

A further object of the present invention relates to the previously mentioned microorganisms which are capable of producing sulfur-containing compounds like L-methionine, L-cysteine, L-homocysteine, L-cystathionine, S-adenosyl-L-methionine, glutathione, biotin, thiamine and/or lipoic acid, preferably L-methionine and/or L-cysteine due to the content/activity, which is reduced as compared to the wild-type, of proteins encoded by nucleic acids that are identical or functionally homologous to nucleic acids having the sequence identified in SEQ ID NO: 1 or due to the content/activity, which is reduced as compared to the wild-type, of nucleic acids that are identical or functionally homologous to nucleic acids having the sequence of SEQ ID NO: 1.

A further object of the present invention relates to microorganisms, in which as compared to the wild-type the content and/or the activity of proteins encoded by nucleic acids that are identical or functionally homologous to nucleic acids having the sequence of SEQ ID NO: 1 has been reduced by means of disruption and/or deletion of the corresponding genomic nucleic acid sequence(s).

A further object of the present invention relates to microorganisms, in which as compared to the wild-type the content and/or the activity of nucleic acids that are identical or functionally homologous to nucleic acids having the sequence identified in SEQ ID NO: 1 or the content and/or the activity of the proteins encoded by said nucleic acids has been reduced by introducing mutations into the genomic nucleic acid sequences leading to the expression of non-functional forms of the proteins encoded by the previously mentioned nucleic acids.

A further object of the present invention relates to microorganisms, in which as compared to the wild-type the content and/or the activity of the nucleic acids that are identical or functionally homologous to nucleic acids having the sequence of SEQ ID NO: 1 has been reduced by means of antisense methods, expression of a ribozyme, which is specific for the mRNA of said nucleic acids, or by expression of ribonuclease P constructs.

A further object of the present invention relates to microorganisms, in which, as compared to the wild-type, the content and/or the activity of the nucleic acids that are identical or functionally homologous to nucleic acids having the sequence SEQ ID NO: 1 or the content and/or the activity of the proteins encoded by said nucleic acids has been reduced by the expression of recombinant antibodies, which are specific for the proteins encoded by the nucleic acids and block or inhibit their activity, in the microorganisms.

A further object of the present invention relates to microorganisms, in which the content and/or activity of the nucleic acids that are identical or functionally homologous to nucleic acids having the sequence SEQ ID NO: 1 or the content and/or the activity of the proteins encoded by said nucleic acids has been reduced, as compared to the wild-type, by overexpressing in the cells a non-functional form of the proteins encoded by said nucleic acids.

A further object of the present invention relates to microorganisms, in which, in addition to the reduction of the content and/or activity of the nucleic acids that are identical or functionally homologous to the nucleic acids having the sequence of SEQ ID NO: 1 or the reduction of the content and/or activity of the proteins encoded by said nucleic acids, the content and/or the activity of nucleic acids coding for McbR from C. glutamicum or for homologs thereof has been reduced as compared to the wild-type.

Thus, an object of the present invention relates to microorganisms, in which, in addition, the content and/or the activity of nucleic acids that are identical or functionally homologous to nucleic acids having the sequence of SEQ ID NO: 3 or the content and/or the activity of proteins that are identical or functionally homologous to proteins having the sequence of SEQ ID NO: 4 has been reduced as compared to the wild-type. Herein, reducing the content or the activity can be performed in the same way as described in the above.

A particularly preferred embodiment of the microorganisms according to the present invention relates to microorganisms, in which the expression of the previously mentioned nucleic acid sequences or proteins encoded thereby is substantially completely suppressed.

A further object of the present invention relates to microorganisms, in which, beside the reduction of the content and/or activity of the nucleic acids that are identical or functionally homologous to nucleic acids having the sequence SEQ ID NO: 1 or of the proteins encoded thereby, the content and/or the activity of at least one further nucleic acid coding for a gene product of the biosynthetic pathway of the desired sulfur-containing compound is increased as compared to the wild-type. In such microorganisms, the last-mentioned nucleic acids can also be mutated in such a way that the protein encoded by the nucleic acid is not influenced with respect to its activity by metabolites of the metabolism. In these microorganisms, the content and/or the activity of McbR from C. glutamicum or of homologs, as mentioned in the above, can additionally be reduced.

In the same manner, a further object of the present invention relates to microorganisms, in which, beside the reduction of the content and/or the activity of nucleic acids that are identical or functionally homologous to nucleic acids having the sequence SEQ ID NO: 1 or of proteins encoded thereby, the content and/or the activity of at least one further nucleic acid coding for a gene product of the biosynthetic pathway of the desired sulfur-containing compound is additionally reduced as compared to the wild-type. In said microorganisms, the content and/or the activity of McbR from C. glutamicum or of homologs as mentioned in the above can additionally be reduced.

A further object of the present invention relates to methods for producing sulfur-containing compounds in microorganisms, which comprise the cultivation of a microorganism according to the present invention. Herein, a particularly preferred method for producing L-methionine and/or L-cysteine employs the microorganism Corynebacterium glutamicum, in which the content and/or the activity of nucleic acids that are identical or functionally homologous to nucleic acids having the sequence SEQ ID NO: 1 or of the proteins encoded by said nucleic acids is reduced as compared to the wild-type.

A further object of the present invention relates to a method for producing L-methionine and/or L-cysteine using microorganisms, wherein, in addition to the reduction of the content and/or the activity of the nucleic acids that are identical or functionally homologous to nucleic acids having the sequence SEQ ID NO: 1 or of the proteins encoded by said nucleic acids, the content and/or activity of further previously mentioned nucleic acids is/are increased or reduced in the manner described.

A further object of the present invention relates to the use of the nucleic acids mentioned in the above for producing microorganisms which can be used for producing sulfur-containing compounds.

Within the scope of the present invention, a novel regulator of methionine biosynthesis could be identified in C. glutamicum. This was made possible using a novel selection and mutation strategy, which significantly differs from the attempts formerly made in the art to identify methionine-overproducing strains of. C. glutamicum (see Examples).

Within the scope of the present invention, it could herein be shown for the first time that the nucleic acid sequence having the SEQ ID NO: 1 (GenBank accession number: NCgl2640) codes for a protein having the amino acid sequence of SEQ ID NO: 2 (GenBank accession number: NP_{—}601931) and being actively involved in the regulation of the biosynthetic pathway of methionine in C. glutamicum. Within the scope of the present invention, it could furthermore be shown for the first time that the deletion or functional disruption of the nucleic acid sequence having the SEQ ID NO: 1 in C. glutamicum leads to strains which produce methionine to an increased extent due to the deactivated regulation of the protein encoded by SEQ ID NO: 1.

A BLAST search at the NCBI showed that homologs of NCgl2640 from C. glutamicum exist in many different microorganisms. As the biosynthetic pathways for sulfur-containing compounds are conserved in many different organisms, it is assumed that microorganisms, in which the content and/or the activity of nucleic acids that are identical or functionally homologous to a nucleic acid having the sequence of SEQ ID NO: 1 is reduced as compared to the wild-type, are also capable of producing methionine and/or other sulfur-containing compounds like cysteine to an increased extent.

According to the present invention, NCgl2640 is understood to denote a nucleic acid molecule having the sequence of SEQ ID NO: 1. The protein encoded by such a nucleic acid molecule has the sequence of SEQ ID NO: 2.

According to the present invention, functional homologs to the nucleic acid having the sequence SEQ ID NO: 1 or to the protein having the amino acid sequence SEQ ID NO: 2 are understood to denote such nucleic acid molecules or proteins, whose sequence exhibits a significant homology to the nucleic acid having the sequence SEQ ID NO: 1 or the protein having the amino acid sequence SEQ ID NO: 2.

According to the present invention, the content of NCgl2640 or the content of nucleic acids that are identical or functionally homologous to a nucleic acid having the sequence of SEQ ID NO: 1 is understood to denote the amount of said nucleic acids or of the proteins encoded by said nucleic acids as can be determined for the respective microorganism.

According to the present invention, the activity of NCgl2640 or of nucleic acids that are identical or functionally homologous to a nucleic acid having the sequence SEQ ID NO: 1 is understood to denote the cellular activity of the proteins encoded by said nucleic acid sequences. The cellular function of proteins encoded by nucleic acids that are identical or functionally homologous to a nucleic acid having the sequence of SEQ ID NO: 1 can be seen in the regulation of the biosynthesis of sulfur-containing compounds and, in particular, of methionine. It can easily be determined whether a nucleic acid homologous to the sequences SEQ ID NO: 1 and 2 has the same activity as the nucleic acid having the sequences SEQ ID NO: 1 or 2 in C. glutamicum. To this end, the respective genomic nucleic acid sequence is deleted in the respective microorganisms and it is determined whether the microorganism is still capable of producing methionine or other sulfur-containing compounds despite the addition of the structural analog of methionine, like for example D,L-ethionine.

According to the present invention, an altered content of nucleic acids compared to the wild-type that are identical or functionally homologous to a nucleic acid having the sequence SEQ ID NO: 1 is understood to denote an amount of said nucleic acids or of the proteins encoded by said nucleic acids that is reduced as compared to the wild-type. The reduction of the amount of said nucleic acids or of the proteins encoded by said nucleic acid is generally achieved by reducing the content of the endogenous nucleic acids which are either nucleic acids having the sequence of SEQ ID NO: 1 or nucleic acids functionally homologous thereto.

According to the present invention, a wild-type is understood to denote the corresponding original organism, which has not been genetically engineered.

The reduction of the activity of the nucleic acids that are identical or functionally homologous to a nucleic acid having the sequence of SEQ ID NO: 1 or of proteins encoded by said nucleic acids can be achieved by reducing the amount or the content of endogenous nucleic acids or of the endogenous proteins encoded by said nucleic acids. Besides, according to the present invention, the reduction of the activity of the mentioned nucleic acids or the proteins encoded by said nucleic acids is understood to denote that the activity of endogenous nucleic acids or of the proteins encoded by said endogenous nucleic acids remains unaltered, while the interaction of the proteins encoded by said nucleic acids with their cellular binding partners, for example by the expression of non-functional forms of NP_{—}601931 or homologs thereof or antibodies specific thereto, is significantly inhibited.

Preferably, the reduction of the content and/or the activity of nucleic acids that are identical or functionally homologous to a nucleic acid having the sequence identified in SEQ ID NO: 1 or of the proteins encoded by said nucleic acids, which occurs in the microorganisms according to the present invention, is at least 5%, preferably at least 10%, especially preferably at least 20%, also especially preferably at least 40%, also especially preferably at least 60%, particularly preferably at least 80%, also particularly preferably at least 90%, also in particular preferably at least 95%, also in particular preferably at least 98%, and most preferably 100%.

According to the present invention, the term “repression of the expression of nucleic acids that are identical or functionally homologous to a nucleic acid having the sequence of SEQ ID NO: 1 or of proteins encoded by said nucleic acids” is synonymous to the term “reduction of the content and/or the activity of said nucleic acids or of proteins encoded by said nucleic acids”.

As has already been mentioned in the above, homologs of NCgl2640 could be identified in many different organisms via a BLAST analysis at the NCBI. As, in the future, more and more sequence data for many different microorganisms will be available, the present invention also includes those microorganisms, in which nucleic acids or proteins encoded thereby are reduced with respect to their content and/or activity as compared to the wild-type, provided that said nucleic acids or the amino acid sequences encoded by said nucleic acid sequences exhibit a significant or substantial sequence homology with the nucleic acids having the sequence of SEQ ID NO: 1 or proteins having the sequence of SEQ ID NO: 2.

Identity of two nucleic acids or proteins is understood to denote the identity of the nucleic acid sequences or amino acid sequences over a particular sequence region, preferably over the entire sequence length, in particular the identity calculated by means of comparison with the aid of the Lasergene software by DNA Star Inc., Madison, Wis. (USA) using the CLUSTAL method (Higgins et al., 1989, Comput. Appl. Biosci., 5 (2), 151). Homologies can also be calculated with the aid of the Lasergene software by DNA Star Inc., Madison, Wis. (USA) using the CLUSTAL method (Higgins et al., 1989), Comput. Appl. Biosci., 5 (2), 151).

Nucleic acid molecules are identical if they have the same nucleotides in the same 5′ to 3′ order.

Amino acid molecules are identical if they have the same amino acids in the same order from the N- to the C-terminus.

According to the present invention, significant or substantial sequence homology is generally understood to denote that the nucleic acid sequence of a DNA molecule or the amino acid sequence of a protein is identical to the nucleic acid or amino acid sequences of SEQ ID NO: 1 or SEQ ID NO: 2, respectively, or their functionally equivalent parts by at least 30%, preferably by at least 40%, also preferably by at least 50%, further preferably by at least 60% or by at least 70%, especially preferably by at least 90%, particularly preferably by at least 95%, and most preferably by at least 98%. Preferably, homology is determined over the entire sequence length of SEQ ID NO: 1 or SEQ ID NO: 2.

Thus, homology is preferably calculated over the entire amino acid or nucleic acid sequence region. Beside the programs mentioned in the above, the person skilled in the art has at his disposal further programs based on different algorithms for comparing different sequences. Herein, the algorithms by Needleman and Wunsch, or Smith and Waterman create particularly reliable results. For sequence comparisons, for example, also the program Pile Aupa (J. Mol. Evolution. (1987), 25, 351-360; Higgins et al., (1989), Cabgos, 5, 151-153) can be used or the programs Gap and Best Fit (Needleman and Wunsch, (1970), J. Mol. Biol., 48, 443-453 and Smith and Waterman (1981), Adv., Appl. Math., 2, 482-489), which are contained in the GCG Software Package by the Genetics Computer Group (575 Science Drive, Madison, Wis., USA 53711), can also be employed.

Significant or substantial homology or sequence homology is also understood to denote that said sequences are functionally homologous to the sequences having the SEQ ID NOS: 1 and 2.

One embodiment of the present invention relates to the use of the previously mentioned nucleic acids for producing microorganisms which are capable of producing increased quantities of sulfur-containing compounds and, in particular, of methionine and/or cysteine as compared to the wild-type. Herein, the person skilled in the art is aware of the fact that, for generating a microorganism capable of producing increased amounts of sulfur-containing compounds, he will in each case reduce the content or the activity, as compared to the wild-type, of the nucleic acid corresponding to the functional homolog of NCgl2640 from C. glutamicum in the respective organism.

DNA sequences having a high homology, i.e. a high similarity or identity, are bona fide candidates for DNA sequences corresponding to the sequences identified in SEQ ID NO: 1 of the nucleic acids according to the present invention. Said gene sequences can be isolated via standard techniques, like for example PCR and hybridization, and their function can be determined by the person skilled in the art by corresponding enzyme activity tests and other experiments. According to the present invention, homology comparisons with DNA sequences can also be employed in order to design PCR primers by identifying at first the regions that are most conserved in the DNA sequences of different organisms. Such PCR primers can then be used to isolate, in a first step, DNA fragments that are components of nucleic acids that are homologous to the nucleic acids of the present invention.

There are a variety of search engines, which can be used for such homology comparisons or searches. Said search engines comprise, for example, the CLUSTAL program group of the BLAST program, which is provided by the NCBI.

Furthermore, a variety of experimental methods, by which DNA sequences can be isolated from most diverse organisms that are homologous to the sequences according to the present invention, are known to the person skilled in the art. Among said methods are, for example, the preparation and screening of cDNA libraries with correspondingly degenerate probes.

Sequences homologous to SEQ ID NO: 2 can, for example, be sequences coding for the following proteins:

NP_{—}601931.1; NP_{—}739184.1; NP_{—}940366.1; NP_{—}962856.1; ZP_{—}00226250.1; NP_{—}214947.1; NP_{—}334857.1; ZP_{—}00058595.1; NP_{—}925240.1; NP_{—}969100.1; ZP_{—}00019148.1; ZP_{—}00199594.1; ZP_{—}00015952.1; ZP_{—}00160492.1; NP_{—}770404.1; NP_{—}866130.1; ZP_{—}00026378.1; NP_{—}280238.1; ZP_{—}00029745.1; ZP_{—}00226340.1; NP_{—}882643.1; NP_{—}459575.1; NP_{—}879442.1; ZP_{—}00213193.1; ZP_{—}00221250.1; NP_{—}806026.1; NP_{—}521417.1; NP_{—}455160.1; ZP_{—}00185946.2; ZP_{—}00203540.1; NP_{—}415113.1; NP_{—}752598.1; NP_{—}286306.1; NP_{—}902574.1; ZP_{—}00052011.1; NP-706431.1; NP_{—}822174.1; NP_{—}745396.1; NP_{—}739496.1 (in each case, these numbers concern the GenBank accession numbers).

As has already been mentioned, altering the content and/or the activity of nucleic acids that are identical or functionally homologous to a nucleic acid having the sequence SEQ ID NO: 1 or of proteins encoded by said nucleic acids can be performed in different ways. Reducing the activity or the content can, for example, be performed by deactivating stimulating regulatory mechanisms on transcriptional, translational and/or protein level or by reducing the gene expression of the respective nucleic acids.

In a preferred embodiment, the expression of the respective genomic sequences will be reduced. Preferably, these will be microorganisms, in which the genomic sequences for the nucleic acids that are identical or functionally homologous to a nucleic acid having the sequence SEQ ID NO: 1 are inactivated. Inactivation of said genomic sequences can be performed differently. One possibility is to delete the genomic sequences by homologous recombination. Another possibility is to introduce insertions into the genomic sequences which in turn prevent the expression of the nucleic acids or lead to non-functional proteins.

Within the scope of the present invention, non-functional proteins are understood to denote such proteins or protein fragments in which the structure, and thus the function of the protein having the amino acid sequence SEQ ID NO: 2 or the proteins functionally homologous thereto, are altered by point mutations, insertions, or deletions in such a way that these proteins do not function any longer as central regulatory switches in the biosynthetic pathways of sulfur-containing compounds and, in particular, of methionine.

It can easily be determined whether non-functional proteins or protein fragments are obtained by introducing insertion, deletion, or mutation into the genomic sequences of nucleic acids that are identical or functionally homologous to a nucleic acid having the sequence of SEQ ID NO: 1. To this end, the microorganisms expressing the putative non-functional proteins or protein fragments are tested for their capability of growing in the presence of structural analogs of methionine, like for example D,L-ethionine. If this is the case, they are non-functional proteins or protein fragments.

The reduction of the expression of the genomic sequences being identical or functionally homologous to SEQ ID NO: 1 by deletion of the genomic sequences or by introduction of mutations, insertions, or deletions leading to non-functional proteins or protein fragments is generally referred to as functional disruption of the genomic sequences.

Herein, the deletion or functional disruption of genomic segments by homologous recombination can be performed by a method comprising the following steps:
a) Producing a vector, comprising the following nucleic acid sequences in 5′ to 3′ orientation:
   a promoter sequence functional in microorganisms,
   operatively linked thereto a DNA sequence being identical or homologous to the 5′ end of the nucleic acid sequence that is identical or functionally homologous to the sequence having the SEQ ID NO: 1,
   operatively linked thereto a DNA sequence coding for a resistance gene,
   operatively linked thereto a DNA sequence being identical or homologous to the 3′ end of the nucleic acid sequence that is identical or functionally homologous to the sequence having the SEQ ID NO: 1,
   operatively linked thereto a termination sequence functional in micro-organisms; and
b) transferring the vector from a) to the microorganism and, optionally, integrating the vector into its genome.

One possibility for homologous recombination can be taken from the Examples given below. It is known to the person skilled in the art that conventional plasmids, as are known, for example, for overexpression of nucleic acid sequences in microorganisms, can be used as vectors for such a homologous recombination. Typically, resistances to antibiotics will be used as resistance genes (see below).

An operative link is understood to denote the sequential arrangement of promoter, coding sequence, terminator, and, optionally, further regulatory elements in such a way that each of the regulatory elements is capable of duly fulfilling its function in the expression of the coding sequence. Examples for operatively linkable sequences are activation sequences, enhancers, and the like. Further regulatory elements comprise selectable markers, amplification signals, replication origins and the like. Suitable regulatory sequences are, for example, described in Goeddel, Gene Expression Technology: Methods in Enzymology 185, Academic Press, San Diego, Calif. (1990).

If point mutations, insertions or deletions leading to non-functional proteins or protein fragments are supposed to be introduced into the genomic sequences, this is conventionally performed by techniques known in the prior art (see, inter alia, Sambrook et al., Molecular Cloning: A Laboratory Manual, (2001), 3^{rd} edition, Cold Spring Harbor Laboratory Press, Cold Spring Harbor, USA and the so-called Quikchange mutagenesis method by Stratagene, La Jolla, USA). Further methods for introducing point mutations, insertions or deletions into genomic sequences of microorganisms are, inter alia, described in Jager et al., (1992) J. Bacteriol. 174, 5462-5465 and in WO 02/070685.

A further possibility of reducing the content and/or the activity of the previously mentioned nucleic acids can be, for example, by an antisense strategy. To this end, for example, a method can be used, which comprises the following steps:
a) Producing a vector, comprising the following nucleic acid sequences in 5, to 3′ orientation:
   a promoter sequence functional in the respective microorganism,
   operatively linked thereto an antisense sequence to SEQ ID NO: 1 or a functional homolog thereof,
   operatively linked thereto a termination sequence functional in the respective microorganism; and
b) transferring the vector from a) to the microorganism and, optionally, integrating the vector into the genome of said microorganism.

In a further embodiment, vectors containing a DNA sequence coding for a ribozyme, which specifically recognizes the mRNA of the nucleic acids that are identical or functionally homologous to a nucleic acid having the sequence of SEQ ID NO: 1, are used for the production of the microorganisms according to the present invention. It is known to one skilled in the art how ribozymes having endonuclease activity directed against a specific mRNA can be produced. This is described in detail, for example, in Steinecke et al., (Steinecke et al. (1992) EMBO J., 11, 1525). Within the scope of this invention, the term ribozyme is also understood to denote such RNA sequences which, beside the actual ribozyme, also comprise leader sequences complementary to the mRNA of the nucleic acids that are identical or functionally homologous to a nucleic acid having the sequence of SEQ ID NO: 1, thus leading to the result that the mRNA-specific ribozyme is directed to the mRNA substrate of the ribozyme even more targetly.

Such a method comprises, for example, the following steps:
a) Producing a vector, comprising the following nucleic acid sequences in 5′ to 3′ orientation:
   a promoter sequence functional in the respective microorganism,
   operatively linked thereto a DNA sequence coding for a ribozyme that specifically recognizes the mRNA of a nucleic acid of the SEQ ID NO: 1 or functional homologs thereof,
   operatively linked thereto a termination sequence functional in the respective microorganism; and
b) transferring the vector from a) to the microorganism and, optionally, integrating the vector into its genome.

A further alternative for the production of microorganisms of the present invention is provided by the transfer of nucleic acids by vectors, comprising a DNA sequence consisting of antisense sequences of the nucleic acids that are identical or functionally homologous to a nucleic acid having the sequence identified in SEQ ID NO: 1 and a sequence coding for RNase P. In the transcription of such vectors RNA molecules are synthesized in the cell, which contain a leader sequence (the antisense sequence) directing the RNase P to the mRNA of the nucleic acids that are identical or functionally homologous to a nucleic acid having the sequence of SEQ ID NO: 1, whereby cleavage of the mRNA via RNase P is effected (see also U.S. Pat. No. 5,168,053). Preferably, the leader sequence comprises 10 to 15 nucleotides being complementary to the DNA sequence of the nucleic acids that are identical or functionally homologous to a nucleic acid having the sequence identified in SEQ ID NO: 1.

Such a method can, for example, comprise the following steps:
a) Producing a vector, comprising the following nucleic acid sequences in 5′ to 3′ orientation:
   a promoter sequence functional in the respective microorganism,
   operatively linked thereto a nucleic acid sequence complementary to the sequence of SEQ ID NO: 1 or a functional homolog or parts thereof,
   operatively linked thereto a DNA sequence coding for ribonuclease P,
   operatively linked thereto a termination sequence functional in the respective microorganism; and
b) transferring the vector from a) to the microorganism and, optionally, integrating the vector into the genome of said microorganism.

The term complementarity describes the capability of a nucleic acid molecule of hybridizing with another nucleic acid molecule on the basis of hydrogen bonds between complementary bases. It is known to the person skilled in the art that two nucleic acid molecules do not have to be 100% complementary for being capable of hybridizing with each other. Preferably, a nucleic acid which is supposed to hybridize with another nucleic acid is complementary to the latter by at least 40%, preferably by at least 50%, also preferably by at least 60% or by at least 70%, especially preferably by at least 80%, also especially preferably by at least 90%, particularly preferably by at least 95%, and most preferably by at least 98% or 100%.

Preferably, complementarity degrees like homology and identity degrees are supposed to be determined over the entire length of the protein or the nucleic acid.

Stringent in vitro hybridization conditions are known to the person skilled in the art and can be taken from the literature (see, for example, Sambrook et al., vide supra). The term specific hybridization refers to the circumstance that, under stringent conditions, a molecule preferentially binds to a nucleic acid having a specific nucleic acid sequence, if said nucleic acid having the specific nucleic acid sequence is part of a complex mixture of, for example, DNA or RNA molecules.

The term stringent conditions thus refers to conditions, under which a nucleic acid preferentially binds to a nucleic acid having a target sequence, but not, or at least in a substantially reduced manner, to nucleic acids having different sequences.

Stringent conditions are dependent on the circumstances. Longer sequences hybridize specifically at higher temperatures. In general, stringent conditions are selected in such a way that the hybridization temperature is about 5° C. below the melting point (Tₘ) for the specific sequence at a defined ionic strength and a defined pH value. Tₘ is the temperature (at a defined pH value, a defined ionic strength, and a defined nucleic acid concentration), at which 50% of the molecules, which are complementary to a target sequence, hybridize with said target sequence. Typically, stringent conditions comprise salt concentrations between 0.01 and 1.0 M sodium ions (or ions of another salt) and/or a pH between 7.0 and 8.3. The temperature is at least 30° C. for shorter molecules, for example for those comprising between 10 and 50 nucleotides. In addition, stringent conditions may comprise the addition of destabilizing agents, like for example formamide.

Typical hybridization and washing buffers are of the following composition.

| | |
|---|---|
| Pre-hybridization solution: | 0.5% SDS |
| | 5 × SSC |
| | 50 mM NaPO₄, pH 6.8 |
| | 0.1% Na pyrophosphate |
| | 5 × Denhardt's Reagent |
| | 100 μg/ml salmon sperm |
| Hybridization solution: | Pre-hybridization solution |
| | 1 × 10⁶ cpm/ml probe (5-10 min, 95° C.) |
| 20 × SSC: | 3 M NaCl |
| | 0.3 M sodium citrate |
| | ad pH 7 with HCl |
| 50 × Denhardt's Reagent: | 5 g Ficoll |
| | 5 g polyvinyl pyrrolidone |
| | 5 g Bovine Serum Albumin |
| | ad 500 ml with A. dist. |

A hybridization is conventionally conducted as follows:

| Optional: | washing the blot for 30 min in 1 × SSC/0.1% SDS at | | |
|---|---|---|---|
| | 65° C. | | |
| Pre hybridization: | at least 2 h at 50 to 55° C. | | |
| Hybridization: | overnight at 55 to 60° C. | | |
| Washing: | 5 min | 2 × SSC/0.1% SDS | hybridization temp. |
| | 30 min | 2 × SSC/0.1% SDS | hybridization temp. |
| | 30 min | 1 × SSC/0.1% SDS | hybridization temp. |
| | 45 min | 0.2 × SSC/0.1% SDS | 65° C. |
| | 5 min | 0.1 × SSC | room temp. |

Normally, the nucleic acids transferred in the antisense strategy comprise between 20 and 1,000 nucleotides, preferably between 20 and 750 nucleotides, particularly preferably about 400 to 800 and 500 to 750 nucleotides. However, nucleic acids comprising between 20 and 500 nucleotides, also particularly preferably between 20 and 300 nucleotides, in particular preferred between 20 and 150 nucleotides, also in particular preferred between 20 and 75 nucleotides, and most preferably about 20 to 50 nucleotides, can also be used. It is also possible that the sequences comprise only about 20 or 25 nucleotides.

If nucleic acids, whose transcription in the cell leads to sequences complementary to the mRNA of the nucleic acid having the sequence of SEQ ID NO: 1 or functionally homologous sequences thereof (like for example in the antisense strategy), are transferred to the microorganism, said sequences do not have to be 100% complementary to the mRNA. It is rather sufficient if said sequences have a complementarity of at least 50%, preferably of at least 60%, especially preferably of at least 70%, also especially preferably of at least 80%, particularly preferably of at least 90%, and most preferably of at least 95%. Herein, deviations may have been caused by deletion, substitution and/or insertion.

In general, it applies that only such complementary sequences can be used in accordance with the present invention, which are capable of specifically hybridizing with mRNA regions of the sequence identified in SEQ ID NO: 1 or functionally homologous sequences thereof. Sequences hybridizing in vivo with RNA regions of proteins other than NP_{—}601931 or proteins homologous thereto and causing the repression of the latter are not suitable for the methods according to the present invention.

Some of the previously mentioned methods can also be conducted with sequences that are not components of the coding part of the mRNA of nucleic acids having the sequence of SEQ ID NO: 1 or complementary thereto. It can, for example, be sufficient if said sequences are sequences from the 5′ or 3′ untranslated region, provided that these regulatory sequences are characteristic for the mRNA of the nucleic acid having the sequence of SEQ ID NO: 1 or the sequences homologous thereto.

Such sequences can, in particular, be employed in case the translation of a mRNA is inhibited by antisense constructs. Thus, according to the present invention, the term mRNA does not only comprise the coding components of the mRNA of the nucleic acid having the sequence of SEQ ID NO: 1 or sequences homologous thereto, but also all regulatory sequences occurring in pre-mRNA or mature mRNA that are characteristic for the mRNA of the nucleic acid having the sequence of SEQ ID NO: 1 or the sequences homologous thereto. Correspondingly, this also applies to the DNA sequence. This relates to, for example, 5′ and 3′ untranslated regions, promoter sequences, upstream activating sequences, etc.

If vectors are employed, whose transcription leads to RNA molecules consisting of a leader sequence and RNAse P, then the leader sequence has to be sufficiently complementary in order to specifically recognize the mRNA of the nucleic acid having the sequence of SEQ ID NO: 1 or the homologous sequences. According to the respective requirements, it can be selected which region of the mRNA will be recognized by the leader sequence. Preferably, such leader sequences comprise about 20 nucleotides; they should, however, not be significantly shorter than 15 nucleotides. With a 100% complementarity of the leader sequence, 12 nucleotides should also be sufficient. Of course, the leader sequences can comprise up to 100 nucleotides or more, as this will only increase their specificity for the respective mRNA.

If, within the scope of the present invention, sense sequences are referred to, this is understood to denote those sequences corresponding to the coding strand of the genes for NCgl2640 or homologs thereof or comprising parts thereof. Herein, said sequences are the sequence of SEQ ID NO: 1 or functional homologs thereof.

If, within the scope of the present invention, antisense sequences are referred to, this is understood to denote those sequences corresponding to the non-coding DNA strand of the genes for NP_{—}601931 or homologs thereof. Thus, said sequences are complementary to SEQ ID NO: 1 or homologs thereof. Of course, said sequences neither have to be 100% identical to the sequence of the non-coding DNA strand, but they can have the previously mentioned degrees of homology. This is also reflected in the circumstance that antisense sequences complementary to the mRNA of a gene according to definition do not have to be 100% complementary to said mRNA. They can, for example, also be complementary by at least 50%, preferably by at least 60%, particularly preferably by at least 70%, further particularly preferably by at least 80%, in particular preferably by at least 90%, and most preferably by at least 95%, 98%, and/or 100%. As has been explained in the above, it is sufficient if the antisense sequences are capable of specifically hybridizing with the respectively interesting mRNA of nucleic acids having the sequence of SEQ ID NO: 1. Hybridization can occur either in vivo under cellular conditions or in vitro.

Hybridization of an antisense sequence with an endogenous mRNA sequence typically takes place in vivo under cellular conditions or in vitro.

Moreover, the terms sense and antisense are known to the person skilled in the art. Correspondingly, the person skilled in the art of gene expression is also aware of how long the nucleic acid molecules used for repression have to be and which homology or complementarity to the sequences of respective interest they need to have. According to the present invention, antisense sequences, which are not capable of specifically hybridizing with coding sense sequences of NCgl2640 or homologs thereof in vivo and/or in vitro, i.e. which are also capable of hybridizing with the coding sense sequences of other protein classes, cannot be used.

In principle, the antisense strategy can be combined with a ribozyme method. Ribozymes are catalytically active RNA sequences, which, if coupled to the antisense sequences, catalytically cleave the target sequences (Tanner et al., (1999) FEMS Microbiol Rev. 23 (3), 257-75). This can increase the efficiency of an antisense strategy.

Furthermore, a gene repression, but also gene overexpression, is also possible by means of specific DNA-binding factors, for example zinc finger transcription factors.

Furthermore, factors inhibiting the target protein itself can be introduced into a cell. The protein-binding factors can, for example, be aptamers (Famulok et al., (1999) Curr Top Microbiol Immunol. 243, 123-36).

Repression can also be conducted by means of aptamers. Aptamers can be designed in such a way that they specifically bind to NP_{—}601931 and reduce the activity of the proteins by competitive reactions. Usually, the expression of aptamers is conducted via vector-based overexpression. The design, selection, and expression of aptamers are well known to the person skilled in the art (Famulok et al., (1999) Curr Top Microbiol Immunol., 243, 123-36).

For said proteins, specific antibodies can be considered as further protein-binding factors, whose expression in microorganisms causes a reduction of the content and/or the activity of the proteins encoded by nucleic acids that are identical or functionally homologous to a nucleic acid having the sequence of SEQ ID NO: 1. The production of such monoclonal, polyclonal, or recombinant antibodies is performed according to standard protocols (Guide to Protein Purification, Meth. Enzymol. 182, pp. 663-679 (1990), M. P. Deutscher, ed.). The expression of antibodies is also known from the literature (Fiedler et al., (1997) Immunotechnology 3, 205-216; Maynard and Georgiou (2000) Annu. Rev. Biomed. Eng. 2, 339-76).

A further method for producing microorganisms according to the present invention intends to reduce the activity of the endogenous nucleic acids that are identical or functionally homologous to a nucleic acid having the sequence of SEQ ID NO: 1 or of the proteins encoded thereby by expressing non-functional mutants of said nucleic acids or proteins in the microorganisms. Such non-functional mutants or forms are forms of the nucleic acids or of the proteins encoded by the nucleic acid, which are no longer capable, or at least only in a very limited manner, of regulating the biosynthesis of sulfur-containing compounds, and in particular of methionine, in the microorganisms. Such non-functional mutants can have point mutations, insertions and/or deletions. They are particularly useful in the production of microorganisms according to the present invention, in which the content of endogenous nucleic acids or of the proteins encoded by these nucleic acid sequences is not altered, however, but the activity of the endogenous nucleic acids that are identical or functionally homologous to a nucleic acid having the sequence identified in SEQ ID NO: 1 or of the proteins encoded by said nucleic acid sequences is blocked by overexpressing the previously mentioned non-functional mutants.

According to the present invention, such non-functional mutants have substantially the same nucleic acid or amino acid sequences as the wild-type forms, that is, for example, the sequences SEQ ID NOS: 1 and 2. They have, however, point mutations, insertions, or deletions of nucleotides or amino acids at some positions, which have the effect that the non-functional mutants, as opposed to the wild-type forms, are not capable, or only in a very limited manner, of interacting with their cellular binding partners. In this manner, the non-functional mutants compete out the interaction of the wild-type forms with the cellular binding partners and thereby decouple the regulation of the biosynthesis of sulfur-containing compounds.

Such functional or non-functional mutants of nucleic acids according to the present invention having the sequence SEQ ID NO: 1 can easily be identified by the person skilled in the art. The person skilled in the art has at his disposal a variety of techniques allowing the insertion of point mutation(s), insertion(s) or deletion(s) into the nucleic acid sequences that are identical or homologous to the SEQ ID NO: 1 (see, inter alia, Sambrook (2001), Molecular Cloning: A Laboratory Manual, 3rd edition, Cold Spring Harbor Laboratory Press, Jäger et al., (1992) J. Bacteriol. 174, 5462-5465, WO 02/070685 and the so-called Quikchange mutagenesis method by Stratagene, La Jolla, USA). After introduction of the point mutation, insertion and/or deletion, which may also generally be referred to as mutation, the person skilled in the art is, by means of corresponding tests as illustrated in the Examples or known from the prior art, capable of determining whether the mutagenized proteins have retained their normal activity (see also supra).

According to the present invention, the term “non-functional proteins or protein fragments” does not comprise such proteins having no substantial sequence homology to NCgl2640 or homologs thereof on the amino acid or nucleic acid level. Within the scope of the present invention, non-functional mutants are also referred to as inactivated or inactive or dominant negative proteins.

Thus, the functional or non-functional mutants according to the present invention, which bear the previously mentioned point mutation(s), insertion(s) and/or deletion(s), or the functionally equivalent parts are characterized by a substantial sequence homology to NCgl2640.

It can easily be determined by the person skilled in the art, whether a non-functional mutant is a mutant in the sense of the present invention. To this end, the desired mutation, i.e. point mutation, insertion, or deletion, is introduced, for example by homologous recombination or site-directed mutagenesis, into the genomic nucleic acid sequence that is identical or functionally homologous to SEQ ID NO: 1 and it is tested, whether said reduction in the content and/or the activity of the endogenous nucleic acid sequences leads to decoupling of the regulation of the biosynthesis of sulfur-containing compounds, so that said microorganisms produce increased amounts of said sulfur-containing compounds. If this is the case, it is thereby shown that it is a non-functional mutation.

The non-functional mutation determined in this manner can then be introduced into a DNA sequence which is located in a vector and is operatively linked to promoter and terminator sequences functional in microorganisms. Subsequently to a transfer of said vectors into the microorganisms, the non-functional mutants can be overexpressed and they compete out the interaction of the endogenous wild-type sequences or proteins with their cellular binding partners.

Such a method can, for example, comprise the following steps:
a) producing a vector, comprising the following nucleic acid sequences in 5′ to 3′ orientation:
   a promoter sequence functional in microorganisms,
   operatively linked thereto a DNA sequence coding for a dominant negative mutant of NCgl2640 or for a homolog thereof,
   operatively linked thereto a termination sequence functional in microorganisms,
b) transferring the vector from a) to the microorganism and, optionally, integrating the vector into the genome of said microorganism.

It is known to the person skilled in the art how (a) point mutation(s), (an) insertion mutation(s), or (a) deletion mutation(s) can be introduced into the nucleic acid sequences encoding NP 601931 or homologs thereof PCR techniques can be preferred, for example, for introducing point mutations (“PCR technology: Principle and Applications for DNA Amplification”, H. Ehrlich, id, Stockton Press). In addition, examples for introducing point mutations into nucleic acids having the sequence of SEQ ID NO: 1 can be found in the Examples.

According to the present invention, microorganisms of the present invention can also be produced in such a way that, for example, a recombinant antibody is expressed in the microorganism, which specifically competes the interaction of the proteins encoded by the nucleic acids that are identical or functionally homologous to a nucleic acid having the sequence of SEQ ID NO: 1 with other cellular binding partners.

How to isolate and identify such recombinant antibodies against NP_{—}601931 or homologs thereof is known to the person skilled in the art and can be taken from the literature (Harlow et al., 1999, Using antibodies: a laboratory manual, Cold Spring Harbor Laboratory Press).

According to the present invention, recombinant antibodies are understood to denote the different known forms of recombinant antibodies as described, for example, in Skerra et al. (Curr. Opin. Immunol. (1993) 2, 250-262). Herein, the recombinant antibodies of the present invention comprise the so-called Fab fragments, Fv fragments, scFv antibodies, scFv homodimers, which are joined by disulfide bridges, as well as so-called VH chains. The Fab fragments consist of assembled complete light chains and truncated heavy chains, whereas Fv fragments consist of non-covalently linked VH and VL chains. A survey of the fragments and recombinant antibodies mentioned can be found in Conrad et al. (Plant Mol. Biol. (1998) 38, 101-109). The Fab and Fv fragments mentioned are capable of associating with one another in vivo.

As it is possible that this process may not run very efficiently, the use of scFv antibodies is preferred according to the present invention. These antibodies consist of the variable portion of the light chain and the variable portion of the heavy chain, which are fused via a flexible linker peptide. The production of such scFv antibodies has been intensively described in the prior art (see, inter alia, Conrad et al., vide supra; Breitling et al. (1999) Recombinant Antibodies, John Wiley & Sons, New York). The scFv antibodies have the same antigen specificity and activity as normal antibodies; however, they do not have to be assembled in vivo from individual chains like other natural or recombinant antibodies. They are thus in particular suitable for the methods according to the present invention.

In the previously named references, it is illustrated in detail how nucleic acid sequences coding for the scFv antibodies preferred according to the present invention can be isolated and generated by the person skilled in the art.

Conventionally, it is assumed herein that existing hybridoma cell lines produce monoclonal antibodies. Subsequently, the cDNAs coding for the light and the heavy chains of the antibody are isolated and, in a second step, the coding regions for the variable region of the light and the heavy chains are fused together to form one molecule.

A further way of generating recombinant antibodies, which is known to the person skilled in the art, is the screening of libraries of recombinant antibodies (so-called phage display libraries, see also Hoogenboom et al. (2000) Immunology Today 21, 371-378; Winter et al. (1994) Annu. Rev. Immunol. 12, 433-455; De Wildt et al. (2000) Nat. Biotechnol. 18, 989-994). In said method it is possible, by means of procedures known to the person skilled in the art, to enrich, select, and isolate recombinant antibodies directed against a given antigen.

A method for expressing antibodies against proteins that are identical or homologous to proteins having the sequence of SEQ ID NO: 2 can, for example, comprise the following steps:
a) producing a vector, comprising the following nucleic acid sequences in 5′ to 3′ orientation:
   a promoter sequence functional in microorganisms,
   operatively linked thereto a DNA sequence coding for a recombinant antibody which is specific for proteins that are identical or homologous to proteins having the sequence of SEQ ID NO: 2,
   operatively linked thereto a termination sequence functional in microorganisms; and
b) transferring the vector from a) to the microorganism and, optionally, integrating the vector into the genome of said microorganism.

If, within the scope of the present invention, it is said that the content and/or the activity of the nucleic acids that are identical or functionally homologous to a nucleic acid having the sequence identified in SEQ ID NO: 1 or of the proteins encoded by said sequences is reduced as compared to the wild-type, the same circumstance is also described by saying that the functional expression of the nucleic acids is reduced and, preferably, entirely suppressed.

Within the scope of the present invention, the term sulfur-containing compound or fine chemical comprises any chemical compound which contains at least one covalently bound sulfur atom and is accessible via a fermentation method according to the present invention.

According to the present invention, the sulfur-containing compounds comprise, inter alia, L-methionine, L-cysteine, L-homocysteine, L-cystathionine, S-adenosyl-L-methionine, glutathione, biotin, thiamine, and/or lipoic acid, and preferably L-methionine and/or L-cysteine.

According to the present invention, the microorganisms preferably are Actinobacteria, Cyanobacteria, Proteobacteria, Chloroflexus aurantiacus, Pirellula sp. 1, Halobacteria and/or Methanococci, preferably Corynebacteria, Mycobacteria, Streptomyces, Salmonellae, Escherichia coli, Shigella and/or Pseudomonas. Particularly preferred are microorganisms selected from Corynebacterium glutamicum, Escherichia coli, microorganisms of the genus Bacillus, in particular Bacillus subtilis, and microorganisms of the genus Streptomyces.

The term metabolite denotes chemical compounds which are present in the metabolism of organisms as intermediates or also final products and which can also have, beside their capacity as chemical components, a modulating effect on enzymes and their catalytic activity. Herein, it is known from the literature that such metabolites can have both an inhibitory and a stimulating effect on the activity of enzymes (Stryer, Biochemistry, (1995) W.H. Freeman & Company, New York, N.Y., USA). It is also described in the literature that it is possible, by means of measures like mutation of the genomic DNA by UV radiation, ionizing radiation, or mutagenic substances and subsequent selection on specific phenotypes, to produce such enzymes, in which the influence by metabolic metabolites has been altered, in organisms (Sahm et al. (2000), Biological Chemistry 381(9-10): 899-910). Said modified properties can also be achieved by directed measures. Herein, the person skilled in the art is familiar with altering, also in a directed manner, specific nucleotides of the DNA coding for the protein in genes for enzymes in such a way that the protein resulting from the expressed DNA sequence has specific novel properties, like, for example, that the modulating effect of metabolic metabolites is altered as compared to the unaltered protein.

Particularly suitable as microorganisms according to the present invention are coryneform bacteria having, as compared to the wild-type, a reduced content and/or activity of nucleic acids that are identical or functionally homologous to a nucleic acid having the sequence identified in SEQ ID NO: 1 or of proteins encoded by said nucleic acids.

Said microorganisms are capable of producing sulfur-containing fine chemicals, in particular L-methionine from glucose, sucrose, lactose, fructose, maltose, molasses, starch, cellulose, or from glycerol and ethanol. From the genus Corynebacterium, particularly the species Corynebacterium glutamicum is suitable, which is known for its capability of producing L-amino acids.

Examples for suitable strains of coryneform bacteria are those of the genus

Corynebacterium, in particular of the species Corynebacterium glutamicum
(C. glutamicum), like Corynebacterium glutamicum ATCC 13032,
Corynebacterium acetoglutamicum ATCC 15806,
Corynebacterium acetoacidophilum ATCC 13870,
Corynebacterium thermoaminogenes FERM BP-1539, and
Corynebacterium melassecola ATCC 17965,
or of the genus Brevibacterium, like

### Brevibacteriumflavum ATCC 14067,

Brevibacterium lactofermentum ATCC 13869, and
Brevibacterium divaricatum ATCC 14020;
or strains derived therefrom, like
Corynebacterium glutamicum KFCC10065 and
Corynebacterium glutamicum ATCC21608,
which also produce the desired fine chemical or (a) precursor(s) thereof.

The acronym KFCC stands for the Korean Federation of Culture Collection, the acronym ATCC for the American Type Strain Culture Collection.

A further embodiment according to the present invention relates to microorganisms, in which, in addition to the reduction of the content and/or the activity of the nucleic acids that are identical or functionally homologous to a nucleic acid having the sequence SEQ ID NO: 1 or of the proteins encoded by said sequences, the content and/or the activity of nucleic acids that are identical or homologous to a nucleic acid having the sequence SEQ ID NO: 3 or of proteins encoded by said last-mentioned nucleic acids is reduced as compared to the wild-type.

The nucleic acid having the sequence of SEQ ID NO: 3 is a nucleic acid coding for the protein McbR from C. glutamicum having the amino acid sequence of SEQ ID NO: 4. Said protein could be shown to be a transcriptional repressor for genes of the biosynthetic pathways of sulfur-containing compounds and, in particular, of methionine in C. glutamicum (Rey et al., vide supra).

By simultaneous reduction of the content and/or the activity of nucleic acids that are identical or functionally homologous to nucleic acids having the sequences of SEQ ID NOS: 1 and 3, microorganisms are provided, which are in particular suitable for producing sulfur-containing compounds. Said sulfur-containing compounds can be the previously mentioned compounds, wherein methionine and cysteine are particularly preferred. The microorganisms can also be the previously mentioned organisms, wherein C. glutamicum and E. coli are preferred.

For reducing the content and/or the activity of nucleic acids that are identical or functionally homologous to nucleic acids having the sequence identified in SEQ ID NO: 3 or of the proteins encoded by said nucleic acids, the same methods and procedures can be used as have been described in the above for the nucleic acids that are identical or functionally homologous to a nucleic acid having the sequence of SEQ ID NO: 1 or for the proteins encoded by the last-mentioned nucleic acids. Herein, terms like homology, complementarity, functional forms, dominant negative mutants, etc. are to be understood in the same way.

A further embodiment of the microorganisms according to the present invention relates to microorganisms, in which, beside the activity or the content, reduced as compared to the wild-type, of nucleic acids that are identical or functionally homologous to a nucleic acid having the sequence identified in SEQ ID NO: 1 or of proteins encoded by said nucleic acids, the content and/or the activity of at least one further nucleic acid coding for a gene of the biosynthetic pathway of the desired sulfur-containing compounds are reduced as compared to the wild-type.

Said further nucleic acids are preferably selected from the group I comprising:
the gene thrB coding for homoserine kinase (EP 1 108 790; DNA-SEQ ID NO: 3453),
the gene ilvA coding for threonine dehydratase (EP 1 108 790; DNA-SEQ ID NO: 2328),
the gene thrC coding for threonine synthase (EP 1 108 790; DNA-SEQ ID NO: 3486),
the gene ddh coding for meso-diaminopimelate-D-dehydrogenase (EP 1 108 790; DNA-SEQ ID NO: 3494),
the gene pck coding for phosphoenolpyruvate carboxykinase (EP 1 108 790; DNA-SEQ ID NO: 3157),
the gene pgi coding for glucose-6-phosphate-6-isomerase (EP 1 108 790; DNA-SEQ ID NO: 950),
the gene poxB coding for pyruvate oxidase (EP 1 108 790; DNA-SEQ ID NO: 2873),
the gene dapA coding for dihydrodipicolinate synthase (EP 1 108 790; DNA-SEQ ID NO: 3476),
the gene dapB coding for dihydrodipicolinate reductase (EP 1 108 790; DNA-SEQ ID NO: 3477),
the gene lysA coding for diaminopicolinate decarboxylase (EP 1 108 790 A2; DNA-SEQ ID NO: 3451),
the gene (GenBank accession number NCgl1072) coding for glycosyl transferase (GenBank accession number NP_{—}600345), and
the gene (GenBank accession number NCgl2817) coding for lactate dehydrogenase (GenBank accession number NP_{—}602107).

Besides, the latter organisms according to the present invention can, of course, also be reduced, as compared to the wild-type, with respect to the content and/or the activity of the nucleic acids that are identical or functionally homologous to a nucleic acid having the sequence of SEQ ID NO: 3 or to the content and/or the activity of the proteins encoded by said nucleic acids.

Herein, a reduction of the content and/or the activity with respect to the nucleic acid sequences mentioned in group I or with respect to the proteins encoded by said sequences can be achieved in the same manner as has been described in the above, for example, for SEQ ID NOS: 1 to 4. Herein, terms like homology, dominant negative mutant, etc. are to be understood as defined in the above.

All of the hitherto mentioned embodiments of the present invention relate to microorganisms which can be used for producing the previously mentioned sulfur-containing compounds. Herein, microorganisms which can be used for producing methionine and/or cysteine are in particular preferred, wherein said microorganisms can be the previously mentioned microorganisms, and C. glutamicum and E. coli are preferred. Herein, the previously mentioned methods can be employed in order to reduce the content and/or the activity of the previously mentioned nucleic acids.

A further embodiment of the present invention relates to the previously mentioned microorganisms, in which additionally the content and/or the activity of nucleic acids coding for a gene product of the biosynthetic pathway of the sulfur-containing compounds to be produced are increased as compared to the wild-type. Herein, the biosynthetic pathways of sulfur assimilation, methionine metabolism, trehalose metabolism, pyruvate metabolism, cysteine metabolism, aspartate semialdehyde synthesis, glycolysis, anaplerosis, pentose phosphate metabolism, citric acid cycle, or of the amino acid export can, in particular, be considered as metabolic pathways.

In this respect, further embodiments of the present invention relate to microorganisms, in which the content and/or the activity of nucleic acids or of proteins encoded by said nucleic acids is increased, wherein the nucleic acids are selected from group II, comprising
the gene lysC coding for aspartate kinase (EP 1 108 790 A2; DNA-SEQ ID NO: 281),
the gene asd coding for aspartate semialdehyde dehydrogenase (EP 1 108 790 A2; DNA-SEQ ID NO: 282),
the gene gap coding for glycerinaldehyde-3-phosphate dehydrogenase (Eikmanns (1992) Journal of Bacteriology 174: 6076-6086),
the gene pgk coding for 3-phosphoglycerate kinase (Eikmanns (1992), Journal of Bacteriology 174: 6076-6086),
the gene pyc coding for pyruvate carboxylase (Eikmanns (1992), Journal of Bacteriology 174: 6076-6086),
the gene tpi coding for triosephosphate isomerase (Eikmanns (1992), Journal of Bacteriology 174: 6076-6086),
the gene metA coding for homoserine O-acetyltransferase (EP 1 108 790; DNA-SEQ ID NO: 725),
the gene metB coding for cystathionine gamma-synthase (EP 1 108 790; DNA-SEQ ID NO: 3491),
the gene metC coding for cystathionine gamma-lyase (EP 1 108 790; DNA-SEQ ID NO: 3061),
the gene glyA coding for serine hydroxymethyl transferase (EP 1 108 790; DNA-SEQ ID NO: 1110),
the gene metY coding for O-acetylhomoserine sulfhydrylase (EP 1 108 790; DNA-SEQ ID NO: 726),
the gene metF coding for methylenetetrahydrofolate reductase (EP 1 108 790; DNA-SEQ ID NO: 2379),
the gene serC coding for phosphoserine aminotransferase (EP 1 108 790; DNA-SEQ ID NO: 928),
a gene serB coding for phosphoserine phosphatase (EP 1 108 790; DNA-SEQ ID NO: 334, DNA-SEQ ID NO: 467, DNA-SEQ ID NO: 2767),
the gene cysE coding for serine acetyl transferase (EP 1 108 790; DNA-SEQ ID NO: 2818),
the gene hom coding for homoserine dehydrogenase (EP 1 108 790; DNA-SEQ ID NO: 1306), and
the gene metH coding for methionine synthase (EP 1 108 790; DNA-SEQ ID NO: 1663),
the gene metE (GenBank accession number NCgl1094) coding for methionine synthase (GenBank accession number NP_{—}600367),
the gene (GenBank accession number NCgl2473) coding for cysteine synthase (GenBank accession number NP_{—}601760),
the gene (GenBank accession number NCgl2055) coding for cysteine synthase (GenBank accession number NP_{—}601337),
the gene (GenBank accession number NCgl2718) coding for sulfite reductase (GenBank accession number NP_{—}602008),
the gene cysH (GenBank accession number NCgl2717), coding for phosphoadenosine phosphosulfate reductase (GenBank accession number NP_{—}602007),
the gene (GenBank accession number NCgl2715) coding for sulfate adenylyltransferase subunit 1 (GenBank accession number NP_{—}602005),
the gene (GenBank accession number NCgl2716) coding for CysN-sulfate adenylyltransferase subunit 2 (GenBank accession number NP_{—}602006),
the gene (GenBank accession number NCgl2719) coding for ferredoxin NADP reductase (GenBank accession number NP_{—}602009),
the gene (GenBank accession number NCgl2720) coding for ferredoxin (GenBank accession number NP_{—}602010),
the gene (GenBank accession number NCgl1514) coding for glucose-6-phosphate dehydrogenase (GenBank accession number NP_{—}600790), and
the gene (GenBank accession number NCgl2014) coding for fructose-1,6-bisphosphatase (GenBank accession number NP_{—}601294).

Preferably, the increase of the content and/or the activity of the proteins encoded by the nucleic acids of group II, which is effected in the microorganisms according to the present invention, amounts to at least 5%, preferably at least 20%, also preferably at least 50%, particularly preferably at least 100%, also particularly preferably at least the factor 5; it is in particular preferably increased by at least the factor 10, also in particular preferably by at least the factor 50, more preferably by at least the factor 100, and most preferably the factor 1000.

The increase of the content and/or the activity of the proteins encoded by the nucleic acids of group II can be achieved in different manners. Preferably, it will be achieved by introducing said nucleic acid sequences in form of DNA sequences into a vector, which additionally contains a promoter functional in microorganisms, a ribosomal binding site functional in microorganisms, and a terminator functional in microorganisms. This vector is then transferred to the microorganisms and, according to the vector selected, is either replicated extrachromosomally along with the microorganisms or is integrated into the genome of the microorganisms. Herein, substantially the same vectors or plasmids as previously described for reducing the content and/or the activity of specific nucleic acids or of the proteins encoded thereby can be used. The same applies to the selection markers used.

Furthermore, an increase of the content and/or the activity of the proteins encoded by the nucleic acids of group II can also be achieved by expressing functional equivalents or analogs or homologs of said nucleic acids in the microorganisms. With respect to the terms homology, complementarity, etc., it is referred to the above.

Within the scope of the present invention, functional equivalents or analogs of the explicitly disclosed polypeptides are polypeptides different from said polypeptides, which further retain the desired biological activity, like for example substrate specificity.

According to the present invention, functional equivalents are understood to denote, in particular, mutants having an amino acid other than the explicitly mentioned amino acid in at least one of the sequence positions, yet still exhibiting a biological activity as mentioned in the above. Thus, functional equivalents comprise the mutants obtainable by one or more amino acid addition(s), substitution(s), deletion(s) and/or inversion(s), wherein the alterations mentioned can occur at any sequence position, provided they give a mutant having the previously mentioned profile of properties. Functional equivalence is, in particular, also given if the reactivity patterns between mutant and unaltered polypeptide match qualitatively, i.e. for example if identical substrates are converted at different speeds.

Of course, functional equivalents also comprise polypeptides accessible from other organisms, like naturally occurring variants. Areas of homologous sequence regions can, for example, be determined by means of sequence comparison and equivalent enzymes can be determined following the explicit specifications of the present invention.

Functional equivalents also comprise fragments, preferably individual domains or sequence motifs of the polypeptides of the present invention, which, for example, have the desired biological function.

Furthermore, functional equivalents are fusion proteins having one of the previously mentioned polypeptide sequences or equivalents derived therefrom and at least one further heterologous sequence, which is functionally different there from, in functional N- or C-terminal linkage (i.e. without substantial mutual impairment of the function of the fusion protein elements). Examples for such heterologous sequences are signal peptides, enzymes, immunoglobulins, surface antigens, receptors or receptor ligands.

Functional equivalents according to the present invention are homologs of the explicitly disclosed proteins. They are homologous to one of said explicitly disclosed sequences by at least 30%, or about 40%, 50%, preferably at least about 60%, 65%, 70%, or 75%, in particular at least 85%, like for example 90%, 95%, or 99%.

Homologs of the proteins or polypeptides of the present invention can be generated by mutagenesis, for example, via point mutation or truncation of the protein. The term homolog, as used herein, relates to a variant of the protein which functions as agonist or antagonist of the protein activity.

Homologs of the proteins according to the present invention can be identified by screening of combinatorial banks of mutants, like for example truncation mutants. For instance, a varied bank of protein variants can be generated by combinatorial mutagenesis on nucleic acid level, like for example by enzymatic ligation of a mixture of synthetic oligonucleotides. There is a multiplicity of methods that can be used for generating banks of potential homologs from a degenerate oligonucleotide sequence. Chemical synthesis of a degenerate gene sequence can be conducted in a DNA synthesizer and the synthetic gene can then be ligated into a suitable expression vector. The use of a degenerate set of genes allows the provision of all the sequences in a mixture which encode the desired set of potential protein sequences. Methods for the synthesis of degenerate oligonucleotides are known to the person skilled in the art (for example Narang al. (1983) Tetrahedron 39, 3; Itakura et al. (1984) Annu. Rev. Biochem. 53, 323; Itakura et al., (1984) Science 198, 1056; Ike et al. (1983) Nucleic Acids Res. 11, 477). In addition, banks of fragments of the protein codon can be used in order to generate a varied population of protein fragments for screening and subsequent selection of homologs of a protein according to the present invention. In one embodiment, a bank of coding sequence fragments can be generated by treating a double-stranded PCR fragment of a coding sequence with a nuclease under conditions wherein nicking only occurs about once per molecule; denaturing the double-stranded DNA; renaturing the DNA, thereby forming double-stranded DNA which can comprise sense/antisense pairs of different nicked products; removing single-stranded portions from newly formed duplexes by treatment with S1 nuclease and ligating the resulting fragment bank into an expression vector. By said method, an expression bank can be derived that encodes N-terminal, C-terminal and internal fragments in different sizes of the protein according to the present invention.

In the prior art, several techniques are known for screening gene products of combinatory banks, which have been generated by point mutations or truncation, and for screening of cDNA banks for gene products having a selected property. Said techniques can be adapted to the fast screening of the gene banks, which have been generated by combinatorial mutagenesis of homologs according to the present invention. The most frequently used techniques for screening large gene banks subjected to high-throughput analysis comprise cloning the gene bank into replicable expression vectors, transforming the suitable cells with the resulting vector bank; and expressing the combinatorial genes under conditions wherein the detection of the desired activity and the isolation of the vector coding the gene whose product has been detected are simplified. Recursive ensemble mutagenesis (REM), a technique increasing the occurrence of functional mutants in the banks, can be used along with the screening tests for identifying homologs (Arkin et al. (1992) PNAS 89, 7811-7815; Delgrave et al. (1993) Protein Engineering 6(3), 327-331).

The metH genes coding, for example, for the enzyme methionine synthase (EC 2.1.1.13) from the organisms of the previously mentioned group II can be isolated as should actually be known.

For isolating the metH genes or also other genes of the organisms from the previously mentioned group II, a gene bank of said organism is first established in E. coli. Establishing gene banks is described in detail in generally known textbooks and manuals. As examples, there are to be mentioned the textbook by Winnacker: “Gene und Klone, Eine Einführung in die Gentechnologie” (Verlag Chemie, Weinheim, Germany, 1990), or the manual by Sambrook et al., vide supra. A well-known gene bank is that of the E. coli K-12 strain W3110, which has been established by Kohara et al. (1987, Cell 50, 495-508) in λ-vectors.

For generating a gene bank of enzymes, for example, of the group II in E. coli, cosmids like the cosmid vector SuperCos I (Wahl et al., (1987) PNAS, 84, 2160-2164), but also plasmids like pBR322 (BoliVal et al. (1979) Life Sciences, 25, 807-818 (1979)) or pUC9 (Vieira et al. (1982), Gene, 19, 259-268) can be employed. Such E. coli strains which are defective with respect to restriction and recombination are particularly suitable as hosts. An example for this is the strain DH5αmcr, which has been described by Grant et al. (1990) PNAS, 87, 4645-4649). The long DNA fragments cloned with the aid of cosmids can subsequently again be subcloned into conventional vectors suitable for sequencing, and can then be sequenced like is described, for example, in Sanger et al. (1977, PNAS, 74, 5463-5467).

The DNA sequences obtained can then be examined by known algorithms or sequence analysis programs, like, for example, the one by Staden (1986, Nucleic Acids Research 14, 217-232), the one by Marck (1988, Nucleic Acids Research 16, 1829-1836) or the GCG program by Butler (1998, Methods of Biochemical Analysis 39, 74-97).

The person skilled in the art can find instructions on identifying DNA sequences by hybridization, inter alia, in the manual “The DIG System Users Guide for Filter Hybridization” by Boehringer Mannheim GmbH (Mannheim, Germany, 1993) and in Liebl et al. (1991, International Journal of Systematic Bacteriology, 41, 255-260). The person skilled in the art can find instructions for amplifying DNA sequences with the aid of the polymerase chain reaction (PCR), inter alia, in the manual by Gait: Oligonucleotide synthesis: A Practical Approach (IRL Press, Oxford, UK, 1984) and in Newton and Graham: PCR (Spektrum Akademischer Verlag, Heidelberg, Germany, 1994).

It is furthermore known that alterations at the N- and/or C-terminus of a protein do not substantially impair but may even stabilize its function. Information thereto can be found by the person skilled in the art, inter alia, in Ben-Bassat et al. (1987, Journal of Bacteriology 169, 751-757), in O'Regan et al. (1989, Gene 77, 237-251), in Sahin-Toth et al. (1994, Protein Sciences 3, 240-247), in Hochuli et al. (1988, Biotechnology 6, 1321-1325), and in known textbooks of genetics and molecular biology.

Plasmids that are replicated in coryneform bacteria are suitable as plasmids for all of the previously mentioned methods for increasing or reducing the content and/or the activity of nucleic acids or of proteins encoded thereby. Numerous known plasmid vectors, like for example pZ1 (Menkel et al. (1989, Applied and Environmental Microbiology 64, 549-554)), pEKEx1 (Eikmanns et al., (1991, Gene, 102, 93-98)), or pHS2-1 (Sonnen et al. (1991, Gene, 107, 69-74)), are based on the cryptic plasmids pHM1519, pBL1, or pGA1. Other plasmid vectors, like for example pCLiK5MCS, or such plasmid vectors based on pCG4 (U.S. Pat. No. 4,489,160) or pNG2 (Serwold-David et al. (1990) FEMS Microbiology Letters 66, 119-124) or pAG1 (U.S. Pat. No. 5,158,891), can be employed in the same manner.

Furthermore, such plasmid vectors, with the aid of which it is possible to employ the method of gene amplification by integration into the chromosome, as has been described, for example, by Reinscheid et al. (1994, Applied and Environmental Microbiology, 60, 126-132) for duplicating or amplifying the hom-thrB operon, are also suitable. In said method, the entire gene is cloned into a plasmid vector which is capable of replicating in a host (typically in E. coli), but not in C. glutamicum. For instance, pSUP301 (Sirnon et al. (1983), Biotechnology 1, 784-791), pK18mob or pK19mob (Schäfer et al., Gene 145, 69-73), (Bernard et al. (1993, Journal of Molecular Biology, 234, 534-541)), pEM1 (Schrumpf et al. (1991), Journal of Bacteriology 173, 4510-4516), or pBGS8 (Spratt et al. (1986), Gene 41, 337-342) can be considered as vectors. The plasmid vector containing the gene to be amplified is subsequently transferred into the desired strain of C. glutamicum by transformation. Methods for transforming are, for example, described in Thierbach et al. (1988, Applied Microbiology and Biotechnology 29, 356-362)), Dunican et al (1989, Biotechnology 7, 1067-1070) and Tauch et al. (1994, FEMS Microbiological Letters 123, 343-347).

For expression, the recombinant nucleic acid construct or gene construct is inserted into a suitable host organism, preferably into a host-specific vector, which allows optimal expression of the genes in the host. Vectors are well known to the person skilled in the art and can, for example, be taken from “Cloning Vectors” (Pouwels P. H. et al., Ed., Elsevier, Amsterdam-New York-Oxford, 1985). Beside plasmids, all other vectors known to the person skilled in the art, like for example phages, transposons, IS elements, phasmids, cosmids, and linear or circular DNA are also understood as vectors. In the host organism, said vectors can be replicated autonomously or chromosomally.

Various antibiotics, like for example chloroampicillin, kanamycin, gentamycin, G418, bleomycin, hygromycin, etc., are suitable as selection markers.

In a preferred embodiment of the present invention, these are microorganisms, in which the nucleic acids encoding the proteins the content and/or the activity of which is to be reduced as compared to the wild-type have been removed from the genome of the microorganisms by homologous recombination or have been altered by homologous recombination in such a way that said nucleic acid sequences are no longer functionally expressed.

Said microorganisms, which are preferred according to the present invention, are thus inactivated with respect to the genomic nucleic acids that are identical or functionally homologous to nucleic acids having the sequences of SEQ ID NO: 1, SEQ ID NO: 3 and to the sequences named in group I by homologous recombination, so that a functional expression of the proteins encoded by the previously mentioned nucleic acid does not occur anymore.

In a further preferred embodiment of the present invention, these are microorganisms additionally overexpressing nucleic acids that are identical or homologous to the nucleic acids of group II or functional equivalents thereof.

In order to achieve overexpression, the person skilled in the art can take different measures, individually or in combination. Thus, the number of copies of the corresponding genes can be increased, or the promoter and regulator region or the ribosome-binding site located upstream of the structural gene can be mutated. Expression cassettes inserted upstream of the structural gene function in the same way. With the aid of inducible promoters, it is additionally possible to enhance expression during the course of fermentative L-methionine production. Expression is also improved by means of measures for prolonging the lifespan of the mRNA. Moreover, enzyme activity is also enhanced by preventing the degradation of the enzyme. The genes or gene constructs can either be present in plasmids with different numbers of copies or they can be integrated and amplified within the chromosome. Alternatively, overexpression of the relevant genes by altering media composition and culture procedure can furthermore be achieved.

The person skilled in the art can find instructions thereto, inter alia, in Martin et al. (1987, Biotechnology 5, 137-146), in Guerrero et al. (1994, Gene 138, 35-41), in Tsuchiya et al. (1988, Biotechnology, 6, 428-430), in Eikmanns et al. (1991, Gene, 102, 93-98), in EP 0 472 869, in U.S. Pat. No. 4,601,893, in Schwarzer et al. (1991, Biotechnology 9, 84-87), in Remscheid et al. (1994, Applied and Environmental Microbiology, 60, 126-132), in LaBarre et al. (1993, Journal of Bacteriology 175, 1001-1007), in WO 96/15246, in Malumbres et al. (1993, Gene 134, 15-24), in JP-A-10-229891, in Jensen et al. (1998, Biotechnology and Bioengineering, 58, 191-195), in Makrides (1996, Microbiological Reviews, 60, 512-538), as well as in known textbooks of genetics and molecular biology.

Examples for promoters suitable for methods employed within the scope of the present invention comprise the promoters groES, sod, eftu, ddh, amy, lysC, dapA, lysA from Corynebacterium glutamicum, but also the gram-positive promoters SPO2, like described in Bacillus subtilis and Its Closest Relatives, Sonenshein, Abraham L., Hoch, James A., Losick, Richard; ASM Press, District of Columbia, Washington and Patek M., Eikmanns B. J., Patek J., Sahm H., Microbiology 1996, 142 1297-1309, but also, however, cos-, tac-, trp-, tet-, trp-tet-, lpp-, lac-, lpp-lac-, lacIq-, T7-, T5-, T3-, gal-, trc-, ara-, SP6-, l-PR- or the l-PL-promoter, which are advantageously employed in gram-negative bacteria. The use of inducible promoters, like for example light- and, in particular, temperature-inducible promoters, like the PᵣPₗ-promoter, is also preferred. In principle, all natural promoters with their regulatory sequences can be used. Moreover, synthetic promoters can also be advantageously used. Further examples for promoters are described in Patek M. et al., J. Biotechnol. 2003, 104, 311-323.

The previously mentioned regulatory sequences are supposed to enable the directed expression of the nucleic acid sequences and, where desired, the expression of the proteins. According to the host organism, this can, for example, mean that the gene is expressed or overexpressed only after induction or that it is expressed and/or overexpressed at once. Herein, the regulatory sequences or factors can positively influence and thereby increase or reduce expression. Thus, enhancement of the regulatory elements can advantageously be performed on transcriptional level using strong transcription signals like promoters and/or enhancers. Besides, enhancement of translation is also possible by, for example, improving the stability of the mRNA.

To this end, common recombination and cloning techniques are employed, like for example those described in the Current Protocols in Molecular Biology, 1993, John Wiley & Sons, Incorporated, New York, N.Y., PCR Methods, Gelfand, David H., Innis, Michael A., Sninsky, John J. 1999, Academic Press, Incorporated, California, San Diego, PCR Cloning Protocols, Methods in Molecular Biology Ser., Vol. 192, 2^{nd} ed., Humana Press, New Jersey, Totowa. Sambrook et al., vide supra and in T. J. Silhavy, M. L. Berman and L. W. Enquist, Experiments with Gene Fusions, Cold Spring Harbor Laboratory, Cold Spring Harbor, N.Y. (1984) and in Ausubel, F. M. et al., Current Protocols in Molecular Biology, Greene Publishing Assoc. and Wiley Interscience (1987).

The present invention also relates to methods for producing sulfur-containing compounds which preferably are the previously mentioned sulfur-containing compounds and, in particular, methionine and/or cysteine. Said methods are characterized in that they utilize the microorganisms of the present invention and that the sulfur-containing compounds are preferably enriched in the cells or in the cell medium by fermentative cultivation of these microorganisms. Herein, it is known to the person skilled in the art that further increases in yield can be achieved by optimizing the preferred fermentation methods. In the methods according to the present invention, the microorganisms produced according to the present invention can be cultivated continuously or discontinuously using the batch method or the fed batch or repeated fed batch method for producing sulfur-containing compounds, in particular L-methionine. A summary of known cultivation methods can be found in the textbook by Chmiel (Bioprozeβtechnik 1. Einführung in die Bioverfahrenstechnik (Gustav Fischer Verlag, Stuttgart, Germany, 1991)) or in the textbook by Storhas (Bioreaktoren und periphere Einrichtungen (Vieweg Verlag, Braunschweig/Wiesbaden, Germany, 1994)).

The culture medium to be used has to suitably meet the requirements of the corresponding strains. Descriptions of culture media for different microorganisms are contained in the manual “Manual of Methods for General Bacteriology” by the American Society for Bacteriology (Washington D.C., USA, 1981).

Said media employable according to the present invention usually comprise one or more carbon sources, nitrogen sources, inorganic salts, vitamins and/or trace elements.

Preferred carbon sources are sugars like mono-, di- or polysaccharides. Particularly suitable carbon sources are, for example, glucose, fructose, mannose, galactose, ribose, sorbose, ribulose, lactose, maltose, sucrose, raffinose, starch, or cellulose. Sugar can also be added to the media via complex compounds like molasses or other side products of sugar refinement. It can also be advantageous to add mixtures of different carbon sources. Further possible carbon sources are oils and fats like, for example, soy oil, sunflower oil, peanut oil and coconut oil, fatty acids like, for example, palmitic acid, stearic acid, or linolic acid, alcohols like, for example, glycerin, methanol or ethanol, and organic acids like, for example, acetic acid or lactic acid.

Nitrogen sources usually are organic or inorganic nitrogen compounds or materials containing said compounds. Exemplary nitrogen sources comprise ammonia gas or ammonia salts, like ammonia sulfate, ammonia chloride, ammonia phosphate, ammonia carbonate, or ammonia nitrate, nitrates, urea, amino acids or complex nitrogen sources like “corn steep liquor”, soy flour, soy protein, yeast extract, meat extract and others. The nitrogen sources can be used individually or as a mixture.

Inorganic salt compounds, which can be contained in the media, comprise chloride, phosphor or sulfate salts of calcium, magnesium, sodium, cobalt, molybdenum, potassium, manganese, zinc, copper and iron.

Inorganic sulfur-containing compounds like, for example, sulfates, sulfites, dithionites, tetrathionates, thiosulfates and sulfides, but also organic sulfur compounds like mercaptans and thiols, can be used as sulfur source for producing sulfur-containing fine chemicals, in particular methionine.

Phosphoric acid, potassium dihydrogen phosphate or dipotassium hydrogen phosphate or the corresponding sodium-containing salts can be used as phosphor source.

Chelating agents can be added to the medium in order to keep metal ions in solution. Particularly suitable chelating agents comprise dihydroxyphenols like catechol or protocatechuate or organic acids like citric acid.

The fermentation media used in accordance with the present invention usually contain also other growth factors like vitamins or growth enhancers, among which are, for example, biotin, riboflavin, thiamine, folic acid, nicotinic acid, pantothenate and pyridoxine. Growth factors and salts are often derived from complex media components like yeast extract, molasses, “corn steep liquor” and the like. Furthermore, suitable precursors can be added to the culture medium. The exact composition of the media compounds is strongly dependent on the respective experiment and is selected individually for each specific case. Information on media optimization can be taken from the textbook “Applied Microbiol. Physiology, A Practical Approach” (Ed. P. M. Rhodes, P. F. Stanbury, IRL Press (1997) p. 53-73, ISBN 0 19 963577 3). Growth media are also available from commercial suppliers, like Standard 1 (Merck) or BHI (Brain heart infusion, DIFCO) and the like.

All of the media components are sterilized either by heat (20 min at 1.5 bar and 121° C.) or by sterile filtration. The components can be sterilized either together or, if necessary, separately. All of the media components can be present at the beginning of cultivation or they can, optionally, be added continuously or batchwise.

Normally, the temperature of the culture lies between 15° C. and 45° C., preferably at 25° C. to 40° C., and can be kept constant or can be varied during the experiment. The pH value of the medium should lie within a range of from 5 to 8.5, preferably about 7.0. During the course of cultivation, the pH value for cultivation can be controlled by adding alkaline compounds like sodium hydroxide, potassium hydroxide, ammonia or ammonia water or acidic compounds like phosphoric acid or sulfuric acid. In order to control foam formation, anti-foaming agents like, for example, fatty acid polyglycol esters can be used. For maintaining the stability of plasmids, suitable selectively acting agents, like for example antibiotics, can be added to the medium. In order to maintain aerobic conditions, oxygen or oxygen-containing gas mixtures, like for example ambient air, are brought into the culture. Cultivation is continued until a maximum of the desired product has formed. Normally, this goal is achieved within 10 hours to 160 hours.

The fermentation broths thus obtained containing, in particular, L-methionine usually have a dry mass of 7.5 to 25 weight %.

It is furthermore also advantageous if the fermentation is run under sugar-limited conditions, at least at the end, in particular over at least 30% of its duration, however. This means that during this time the concentration of utilizable sugar in the fermentation medium is kept at or lowered to ≧0 to 3 g/l.

The fermentation broth is subsequently processed. According to the requirements, the biomass can be removed from the fermentation broth completely or partially by separation methods like, for example, centrifugation, filtration, decanting or a combination of said methods, or the entire biomass can remain in the broth.

Subsequently, the fermentation broth can be thickened or reconcentrated by known methods, like for example with the aid of a rotary evaporator, thin film evaporator, drop film evaporator, by reverse osmosis, or by nanofiltration. Said reconcentrated fermentation broth can subsequently be processed by lyophilization, spray drying, spray granulation or by other methods.

It is also possible, however, to further purify the sulfur-containing fine chemicals, in particular L-methionine. The product-containing broth is therefore subjected to chromatography with a suitable resin after removal of the biomass, wherein the desired product or the contaminations are retained on the chromatographic resin entirely or partially. Said chromatographic steps can optionally be repeated, wherein the same or different chromatographic resins are employed. The person skilled in the art is well-versed in selecting suitable chromatographic resins and employing them in the most effective manner. The purified product can be concentrated by filtration or ultrafiltration and stored at a temperature at which the stability of the product is at its maximum.

The identity and purity of the isolated compound(s) can be determined by means of prior art techniques. These comprise high-performance liquid chromatography (HPLC), spectroscopic methods, staining methods, thin film chromatography, NIRS, enzyme test or microbiological tests. Said analysis methods are summarized in: Patek et al. (1994, Appl. Environ. Microbiol. 60, 133-140), Malakhova et al. (1996, Biotekhnologiya 11, 27-32), Schmidt et al. (1998, Bioprocess Engineer. 19, 67-70), Ulmann's Encyclopedia of Industrial Chemistry (1996, Vol. A27, VCH: Weinheim, p. 89-90, p. 521-540, p. 540-547, p. 559-566, 575-581 and p. 581-587); Michal, G (1999, Biochemical Pathways: An Atlas of Biochemistry and Molecular Biology, John Wiley and Sons), Fallon et al. (1987, Applications of HPLC in Biochemistry in: Laboratory Techniques in Biochemistry and Molecular Biology, Vol. 17).

By the methods according to the present invention it is preferably achieved that the production of sulfur-containing compounds, in particular of methionine or cysteine, is increased, as compared to the wild-type, by at least 5%, 10%, 50%, 75%, preferably by at least the factors 2, 3, 4, 5, 7, 10, particularly preferably by at least the factors 20, 40, 60, 80, 100, in particular by the factors 200, 500, 1,000, and most preferably by the factor 10,000. In terms of absolute quantities, in particular a yield of 1 g to 150 g methionine per liter fermentation broth can be achieved by the method of the present invention.

The present invention will now be described in more detail by means of the following non-limiting Examples:

### EXAMPLES

### 1. Materials and methods

### 1.1 Bacterial Strains, Media and Plasmids

C. glutamicum ATCC 14752 or ATCC 13032 were routinely cultivated in CGXII minimal medium (Keilhauer et al. (1993) J. Bacteriol. 175, 5595-5603). The transposon mutants were cultivated in the presence of kanamycin (15 mg ml⁻¹). E. coli DH5α was used for standard cloning and E. coli ET12567 was used for plasmid amplification in case the plasmids were supposed to be transformed in C. glutamicum.

The strains and plasmids used in the present invention are illustrated in Table 1. Luria broth (LB) medium, which had been mixed with corresponding antibiotics (kanamycin 50 μg ml⁻¹, chloramphenicol, 50 μg ml⁻¹, ampicillin, 100 μg ml⁻¹), was used as standard medium for E. coli strains. LB medium, which had been mixed with 4 mM MgSO₄ and 10 mM KCl (Psi broth), was used as recovery medium for chemically transformed E. coli. The recovery medium for electroporated C. glutamicum strains was LBIS (Luria broth with brain heart infusion) and Sorbitol (Liebl et al. (1989) FEMS Microbiol Lett, 65, 299-304). The plasmid pCGL0040 (GenBank accession no. U53587) was used as donor for the transposon Tn5531 (IS1207 Km^{r}) and was amplified in E. coli ET12567.

### 1.2 Recombinant DNA Technology

The transformation of E. coli cells with plasmid DNA was conducted with chemically competent E. coli DH5α or ET12567. The cells were generated according to the rubidium chloride method [http://micro.nwfsc.noaa.gov/protocols/] and transformed as described in Sambrook et al. (vide supra). The generation of competent C. glutamicum cells and electrotransformation were conducted as described in the literature (Ankri (1996), Plasmid, 35, 62-66; Liebl et al., vide supra).

Genomic DNA was isolated using the Wizard® Genomic DNA Purification Kit (Promega). Plasmid preparations from E. coli cells were routinely conducted using the QIAprep Miniprep Kit (Qiagen). Restriction endonucleases were purchased from Roche Diagnostics. Digested DNA fragments were recovered from agarose gel by means of the QIAEX II Gel Extraction Kit (Qiagen). Standard DNA techniques were conducted as described in the literature (Sambrook et al., vide supra). DNA sequencings were conducted using the Global Edition IR2 System (LI-COR Inc., Lincoln, Nebr.).

As genome database for identifying ORFs and promoters, the ERGO database (Integrated Genomics, Chicago, USA) was used. The NCgl numbers given in the text refer to the C. glutamicum ATCC 13032 genomic sequence from the GenBank at the NCBI (GenBank accession No. NC 003450). The promoters for cysteine synthase (NCgl2473, promoter position 2721625-2721822), sulfite reductase (NCgl2718, promoter position 3005188-3005389), and O-acetylhomoserine sulfhydrolase (metY, NCgl0625, promoter position 667771-668107) derived from the ERGO database were amplified by PCR and fused via Xhol and BamHI linkers to promoter-free lacZ in the plasmid pClik (Cm^{r}), which had been cleaved by means of the same restriction enzymes. The plasmids obtained were referred to as pClik-H185, pClik-H187 and pClik-H217 (see Table 1). One individual BglII restriction site in said plasmids was used to introduce NCgl2640, which had been amplified by PCR, wherein the primer pair 2640-fwd-BglII (5′-CCGCTGCTGCTGGTGGCGCTAGATCTGCTAACGGC-3′) and 2640-rev-BglII (5′-ATGTGTTGGGAGATCTCTTAAGTTATTTAGTCCAG-3′) was used. The amplified DNA fragment comprised putative regulatory elements, which were located up to 370 base pairs upstream of the NCgl2640 gene.

### 1.3 Transposing and Mutagenesis, Screening and Localization of the Transpose and Insertion Sites

The plasmid pCGL0040 was isolated from E. coli ET12567. Subsequently, C. glutamicum ATCC 14752 was transformed with the plasmid by electroporation. The transposon insertion mutants were selected via plating on LBHIS, which contained 20 μg kanamycin ml⁻¹. All mutants obtained were pooled, washed twice with sterile 0.9% NaCl, and plated on CGXII medium, which contained kanamycin (20 μg ml⁻¹) and ethionine (7.5 g l⁻¹), in form of 100 μl aliquots of a 106 dilution of the united pool. The fastest growing clone was selected for further analysis.

In order to localize the transposon insertion sites, genomic DNA was isolated from the mutants. The insertion sites were then determined by cloning of the transposon chromosome linker sites into pUC18 and subsequent sequencing with the oligonucleotide Tn5531-Eco (5′-CGGGTCTACACCGCTAGCCCAGG-3′) (Simic et al. (2001) J. Bacteriol. 183, 5317-5324). The sequences thus obtained were then analyzed by means of the BLASTn program against the NCBI GenBank sequence and the ERGO database. Various sequence analysis tools (http://www.expasy.org/, http://npsa-pbil.ibcp.fr oder http://pfam.wustl.edu/ (protein family database PFAM) were employed for pattern and profile searches with the NCgl2640 sequence.

1.4 Chromosomal Deletion of NCgl2640 in C. glutamicum ATCC13032

By means of the two primer pairs

| | |
|---|---|
| 2640-SacB1 | (5′-GAGAGGGCCCATCAGCAGAACCTGGAACC-3′)/ |
| 2640-SacB2 | (5′-GATCCAGAGGTCCACAACC-3′) |
| and | |
| 2640-SacB3 | (5′-GATGGTTCAAGACGAACTCC-3′)/ |
| 2640-SacB4 | (5′-GAGAGTCGACCAGAATCAATTCCAGCCTTC-3′), |

the upstream and downstream region of NCgl2640 was amplified by PCR from chromosomal DNA of C. glutamicum ATCC13032. The fragments obtained were digested with ApaI/XbaI or SpeI/SalI and were jointly cloned into a non-replicative vector pClik-SacB, which was digested with ApaI/SalI, whereby the plasmid pSdel-NCgl2640 was obtained. C. glutamicum ATCC13032 was then transformed by electroporation with the non-replicative plasmid pSdel-NCgl2640. Clones resistant to kanamycin contained the chromosomally integrated plasmids. Subsequently, selection for plasmid loss was conducted by screening for mutants resistant to sucrose according to the method of Schäfer et al. (Schäfer et al. (1994) Gene 145:69-73). Deletion was then verified by PCR analysis and Southern blotting.

### 1.5 LacZ Activity Measurements

Selected mutated strains of C. glutamicum were complemented with the pClik plasmids H185, H187, and H217 or derivatives thereof, which had been complemented with NCgl2640 (see Table 1). The transformants were cultivated in CGXII minimal medium, which contained kanamycin (20 μg ml⁻¹) and chloramphenicol (15 μg ml⁻¹), in the presence or absence of 10 mM L-methionine. Cells were cultivated until the early log phase (OD₆₀₀=1-2) and examined with respect to β-gal activity, as described in Sambrook et al. (Sambrook et al., vide supra). The assays were each performed three times in four independent test series.

### 1.6 Isolating DNA-Binding Proteins

The principle of isolating DNA-binding proteins by means of DNA affinity chromatography using magnetic beads has substantially been described by Gabrielsen et al. (1993), Methods Enzymol., 218, 508-225) and a detailed protocol for C. glufamicum is available, see Rey et al. (vide supra). With a few slight exceptions, the latter protocol was presently employed. With the exception of the elution buffers, all buffers were mixed with 2.5 mM L-methionine. Immediately after cell disruption, the crude extracts were protected against proteolysis by a protease inhibitor (PMSF, aprotinin, leupeptin (Rosenberg et al. (1996) Protein Analysis and Purification. Benchtop Techniques, Boston, Birkhäuser). Subsequently to ultracentrifugation of the crude extract (200,000 g, 40 min, 40° C.), the protein solution was desalted by gel filtration (Sephadex G25). Biotinylated PCR-amplified promoter DNA was immobilized on streptavidin-coated Dynabeads® M270 (Dynal Biotech.). As a negative control fragment, a 460 bp fragment from the upstream region of the groES gene from C. glutamicum was amplified. The washing buffers contained large amounts of unspecific competitor DNA (salmon sperm DNA, 0.4 mg ml⁻¹, Sigma). I D-SDS-PAGE was performed with a 4% stacking gel and a 12% running gel (Schägger et al. (1987) Anal. Biochem. 166, 368-379) and the proteins were stained with colloidal Coomassie Brilliant Blue G-250. The protocol for the tryptic digestion and the MALDI-TOF analysis substantially corresponds to the protocol described in Hermann et al. (2001) Electrophoresis, 22, 1712-1723.

### 1.7 Determining the Extra- and Intracellular Methionine Concentrations

In form of its o-phthaldialdehyde derivative, methionine was quantified by means of HPLC (Molnar-Perl et al. (2001), J. Chromatogr. 913, 283-302). C. glutamicum was cultivated until the stationary phase in 500 ml shake flasks having a culture volume of 50-100 ml at 30° C. and 225 rpm. The cells were removed by centrifugation (10,000 g, 10 min, 4° C.) and the methionine concentration was determined by HPLC analysis. In order to determine the intracellular methionine concentrations, the cells were separated from the liquid and activated by means of silicon oil centrifugation (Ebbighausen et al. (1989) Appl. Microbiol. Biotechnol., 31, 184-190). Subsequently the cells were lysed by means of ultrasonic treatment or by means of a blue-capped ribolyser (FastPrep®, Q-Biogene). The soluble protein in the supernatant was determined according to a Bradford assay. The intracellular content of soluble protein in C. glutamicum was thus empirically determined to be 250 mg ml⁻¹. Based on this value, the total internal cell volume of a sample was calculated in order to be able to determine the methionine concentration following HPLC analysis.

### 2. Results

### 2.1 Selecting and Identifying Ethionine-Resistant Transposon Mutants

Within the scope of the present invention, it was assumed from the hypothesis that an ethionine resistance can be achieved via methionine overproduction, which can in turn be achieved by inactivating putative repressors, which could be involved in the regulation of methionine biosynthesis in C. glutamicum. The reason for this lies in the fact that ethionine is a structural analog of methionine, which cannot be metabolized and thus pretends high concentrations of methionine. As a result, methionine biosynthesis is usually downregulated and the organism finally dies due to a lack of methionine.

One of the possibilities an organism can employ in order to circumvent this toxicity caused by an antimetabolite is to compete out the toxic agent by overproducing the natural metabolite, in this case methionine. In this manner, methionine-resistant strains have already been produced in the 1970s (Kase et al., vide supra). However, no strains significantly overproducing methionine could be obtained at that time, probably because, in the selection experiments performed at that time, no mutants deactivating the biosynthesis of methionine as central regulator had been identified. This was one of the reasons for the present attempts of introducing the mutations by means of transposon-mediated mutagenesis. C. glutamicum ATCC 14752 was thus transformed with pCGL0040 as donor for the transposon Tn5531. In this manner, about 7,000 mutants were obtained on LBHIS plates with kanamycin. Said mutants were, as described in the prior art, pooled and plated on plates containing CGXII medium with 7.5 g D,L-ethionine l⁻¹ plus kanamycin. About 100,000 colony forming units (CFU) were plated in order to ensure that all of the possibly ethionine-resistant mutants could be recovered. With 6 g l¹ ethionine, the growth of the wild-type was inhibited for at least 4 days. After 2 days, 11 kanamycin- and ethionine-resistant mutants could be isolated. All mutants contained the identical Tn5531 insertion in the ORL NCgl2640. Said mutation was referred to as 14752-A2640 and a clone was selected for the following experiments (Table 1).

### 2.2 Sequence Analysis of the Transposon Insertion Sites

It could be observed that the site of transposon insertion in strain 14752::2640 is located in the C-terminal half of the putative protein in the position 2918026/2918027 (GenBank accession number NC 003450). NCgl2640 is separated from NCgl2639 by 7 base pairs, so that both genes are presumably organized in one operon. NCgl2639 is annotated in the database (GenBank) as a putative hydrolase or acetyltransferase (see FIG. 2). NCgl2640 codes for a protein of 42 kDa. Via homology, more than 25 putative bacterial proteins of significant homology (e-value<2e-20) could be identified, none of which has hitherto been annotated in a functional manner. With a search for conserved domains, a consensus pattern for proteins of unknown function (COG2170) could be identified with high significance, just like for motif 04107 of the protein family database (PFAM), which is characteristic for the glutamate cysteine ligase family. No further consensus motifs could be identified. In particular, no DNA-binding proteins could be identified.

### 2.3 Verification of the Methionine-Resistant Phenotype

C. glutamicum ATCC 14752 and the mutated strain were then cultivated in CGXII medium, which contained 3 g l⁻¹ glucose, in the presence or absence of 7.5 g l⁻¹ (mutant) or 3.8 g l⁻¹ (wild-type) D,L-ethionine. The growth of the mutant in the presence of ethionine could neither be discerned from the growth without methionine nor from the growth of the wild-type without ethionine. The growth of the wild-type in the presence of substantially lower sublethal concentrations of ethionine was, however, significantly impaired (see FIG. 3).

In order to exclude that mutations at other sites could have created the ethionine-resistant phenotype, the effect of inactivating NCgl2640 in C. glutamicum ATCC 13032 was tested. NCgl2640 was cut out from the genome of C. glutamicum by homologous recombination and by the selection of sucrose-tolerant mutant strains. Said mutant, which was referred to as 13032::2640, was resistant to D,L-ethionine at 7.5 g/l, whereas no growth of the wild-type ATCC 13032 could be detected on these plates.

Thus, within the scope of the present invention, it could be proven that the ethionine-resistant phenotype is indeed exclusively based on the inactivation of NCgl2640.

### 2.4 Altered Expression of Methionine Biosynthesis Genes in the NCgl2640 Mutant

It was then examined whether the usually strict regulation of sulfur assimilation, which is a key step in methionine biosynthesis, is altered in the NCgl2640 mutant.

It was assumed from the hypothesis that, if NCgl2640 is a central regulator of the biosynthesis of sulfur compounds in C. glutamicum, it regulates the expression of genes involved in the assimilation of sulfur and an expression of increased amounts of methionine should occur, which would be responsible for the resistance to ethionine. Said hypothesis was examined on the basis of the influence of an NCgl2640 knockout on the expression levels of metY, cysK and sulfate operon genes. To this end, the strain 14752::2640, which is deleted with respect to NCgl2640, and the wild-type were transformed with the lacZ reporter plasmids pClik-PcysK, pClik-PmetY and pClik-Psulfat (see also Table 1). The ATCC 14752 wild-type and the mutated strain were then cultivated until the log phase (OD₆₀₀=3) in CGXII medium with or without the addition of 10 mM L-methionine and gene expression was determined by means of lacZ activity.

In the wild-type, the presence of methionine reduced the expression levels of all genes examined. The expression of the sulfate operon was completely suppressed. In the mutated strain, a significant de-repression for cysK and the sulfate operon was observed. In both genes, the expression of methionine was independent, which indicates a complete derepression, i.e. a complete deregulation, of the biosynthetic pathway for methionine and other sulfur-containing compounds in the mutated strain background (see also FIG. 4). In order to exclude polar effects of the inactivation of NCgl2640 on the adjacent NCgl2639 (hydrolase or acetyltransferase, see FIG. 2), NCgl2640 was expressed via its promoter in the mutant, which reestablished the wild-type phenotype, i.e. a methionine-induced repression and reduced (cysK) or increased (sulfate operon) basal transcription levels were observed (see also FIG. 4). Said phenotype is even more developed in the complemented strains than in the wild-type. The reason for this presumably lies in the fact that in the complemented strain the expression of NCgl2640, which results from a medium copy plasmid, is increased.

2.5 NCgl2640 Does Not Bind to The promoters of cysK, metY and the Sulfate Operon

Within the scope of the present invention, it could thus be shown that the expression of cysK, the sulfate operon and also of metY is centrally regulated by NCgl2640. As classical DNA binding motifs could not be identified in the sequence of NCgl2640, a DNA affinity purification in a so-called pull down assay was employed in order to check whether NCgl2640 can bind to the respective promoter regions in the previously mentioned genes. The promoters amplified by PCR and immobilized on beads were incubated in the presence of 2.5 mM L-methionine with crude extracts of C. glutamicum cells, which had been cultivated in the presence or absence of 10 mM L-methionine. Proteins eluting from the promoters at high salt concentration (>200 mM) were separated by 1 D-SDS-PAGE and analyzed by MALDI-TOF.

With a similar approach, Rey et al. (vide supra) had previously identified the McbR repressor together with four other proteins, which appear to specifically bind the metY promoter. These results could be verified within the scope of the present invention. It could furthermore be shown that McbR also binds to the promoters of cysK and to the sulfate operon (see FIG. 5). In both studies, however, NCgl2640 could not be detected, which indicates that no direct DNA/protein interaction is involved in the NCgl2640 mediated regulation.

### 2.6 Increased Amounts of L-Methionine in the NCgl2640 Mutant

The resistance of C. glutamicum 14752::2640 to large amounts of ethionine thus appears to be a result of an increased biosynthesis of L-methionine. In order to verify said hypothesis, the production of L-methionine in the wild-type and in mutants was examined in CGXII minimal medium batch cultures.

It could be shown that the mutated strain usually produced at least twice as much methionine as the wild-type. In the presence of ethionine, the methionine secretion could be stimulated in the mutant, but not in the wild-type (see FIG. 6A). Intracellular amounts of methionine were likewise doubled in the mutant, whereas ethionine reduced the intracellular amount of methionine, which is presumably based on a stimulation of the methionine secretion (see FIG. 6B). The total amount of methionine was twice as high in the mutant as in the wild-type.

The experiments illustrated in the above show that microorganisms, in which the coding sequences for NCgl2640 are deleted and thus the content and/or the activity of protein encoded by said nucleic acid are reduced as compared to the wild-type, are capable of producing methionine in increased amounts.

As the genes of the sulfur metabolism are regulated by NCgl2640, which probably is a trans-acting regulator, the microorganisms according to the present invention can also be employed for producing other sulfur-containing compounds. This is based on the fact that, for example, the microorganisms must also fall back on the metabolic pathways leading to reduction of sulfur for the production of cysteine and that further sulfur-containing compounds, like for example glutathione or S-adenosyl methionine, depend on the presence of cysteine and/or methionine.

Sequence data
SEQ ID NO: 1 (GenBank accession number NCg12640):
ATGGGCATTGAGTTTAAGCGTTCACCGCGACCCACCCTGGGCGTTGAGTG
GGAAATTGCACTTGTTGATCCAGAAACACGTGATCTAGCCCCGCGCGCTG
CAGAAATACTAGAGATTGTGGCCAAGAACCACCCTGAGGTGCACCTCGAG
CGCGAATTCCTCCAAAACACCGTGGAGCTTGTCACCGGAGTGTGCGACAC
CGTCCCCGAAGCGGTGGCAGAGCTTTCCCACGATCTAGATGCGCTGAAAG
AAGCAGCGGATTCTCTCGGGCTTCGGTTGTGGACCTCTGGATCCCACCCA
TTTTCGGATTTCCGCGAAAACCCAGTATCTGAAAAAGGCTCCTACGACGA
GATCATCGCGCGCACCCAATACTGGGGAAACCAGATGTTGATTTGGGGCA
TTCACGTCCACGTGGGCATCAGCCATGAAGATCGCGTGTGGCCGATCATC
AATGCGCTGCTGACAAATTACCCACATCTGTTGGCACTTTCTGCAAGCTC
TCCAGCATGGGACGGACTTGATACCGGTTATGCCTCCAACCGGACGATGC
TCTACCAACAGCTGCCTACAGCCGGACTGCCATACCAATTCCAAAGCTGG
GATGAATGGTGCAGCTACATGGCGGATCAAGATAAATCCGGTGTCATCAA
CCACACCGGATCCATGCACTTTGATATCCGCCCCGCATCCAAATGGGGAA
CCATCGAAGTCCGCGTGGCCGATTCTACCTCCAACCTGCGGGAACTGTCT
GCCATCGTGGCGTTGACCCACTGTCTCGTGGTGCACTACGACCGCATGAT
CGACGCTGGCGAAGAGCTTCCCTCCCTGCAACAATGGCACGTTTCGGAAA
ATAAATGGCGCGCGGCTAGGTATGGTCTGGATGCCGAAATCATCATTTCC
AGAGACACCGATGAAGCGATGGTTCAAGACGAACTCCGCCGACTAGTAGC
GCAATTGATGCCTCTAGCCAACGAACTCGGCTGCGCTCGTGAGCTTGAAC
TTGTGTTGGAAATCCTGGAACGTGGTGGTGGATACGAACGCCAACGCAGA
GTGTTTAAAGAAACTGGCAGTTGGAAAGCTGCAGTTGATTTAGCCTGCGA
CGAACTCAACGACCTCAAAGCACTGGACTAA
SEQ ID NO: 2 (GenBank accession number NP_601931):
MGIEFKRSPRPTLGVEWEIALVDPETRDLAPRAAEILEIVAKNHPEVHLE
REFLQNTVELVTGVCDTVPEAVAELSHDLDALKEAADSLGLRLWTSGSHP
FSDFRENPVSEKGSYDEIIARTQYWGNQMLIWGIHVHVGISHEDRVWPII
NALLTNYPHLLALSASSPAWDGLDTGYASNRTMLYQQLPTAGLPYQFQSW
DEWCSYMADQDKSGVINHTGSMHFDIRPASKWGTIEVRVADSTSNLRELS
AIVALTHCLVVHYDRMIDAGEELPSLQQWHVSENKWRAARYGLDAEIIIS
RDTDEAMVQDELRRLVAQLMPLANELGCARELELVLEILERGGGYERQRR
VFKETGSWKAAVDLACDELNDLKALD
SEQ ID NO: 3 (McbR):
GTGGCTGCTAGCGCTTCAGGCAAGAGTAAAACAAGTGCCGGGGCAAACCG
TCGTCGCAATCGACCAAGCCCCCGACAGCGTCTCCTCGATAGCGCAACCA
ACCTTTTCACCACAGAAGGTATTCGCGTCATCGGTATTGATCGTATCCTC
CGTGAAGCTGACGTGGCGAAGGCGAGCCTCTATTCCCTTTTCGGATCGAA
GGACGCCTTGGTTATTGCATACCTGGAGAACCTCGATCAGCTGTGGCGTG
AAGCGTGGCGTGAGCGCACCGTCGGTATGAAGGATCCGGAAGATAAAATC
ATCGCGTTCTTTGATCAGTGCATTGAGGAAGAACCAGAAAAAGATTTCCG
CGGCTCGCACTTTCAGAATGCGGCTAGTGAGTACCCTCGCCCCGAAACTG
ATAGCGAAAAGGGCATTGTTGCAGCAGTGTTAGAGCACCGCGAGTGGTGT
CATAAGACTCTGACTGATTTGCTCACTGAGAAGAACGGCTACCCAGGCAC
CACCCAGGCGAATCAGCTGTTGGTGTTCCTTGATGGTGGACTTGCTGGAT
CTCGATTGGTCCACAACATCAGTCCTCTTGAGACGGCTCGCGATTTGGCT
CGGCAGTTGTTGTCGGCTCCACCTGCGGACTACTCAATTTAG
SEQ ID NO: 4 (GenBank accession number NP_602128):
MAASASGKSKTSAGANRRRNRPSPRQRLLDSATNLFTTEGIRVIGIDRIL
READVAKASLYSLFGSKDALVIAYLENLDQLWREAWRERTVGMKDPEDKI
IAFFDQCIEEEPEKDFRGSHFQNAASEYPRPETDSEKGIVAAVLEHREWC
HKTLTDLLTEKNGYPGTTQANQLLVFLDGGLAGSRLVHNISPLETARDLA
RQLLSAPPADYSI

### LEGENDS OF THE FIGURES

FIG. 1 The separate way to the incorporation of sulfur in methionine biosynthesis

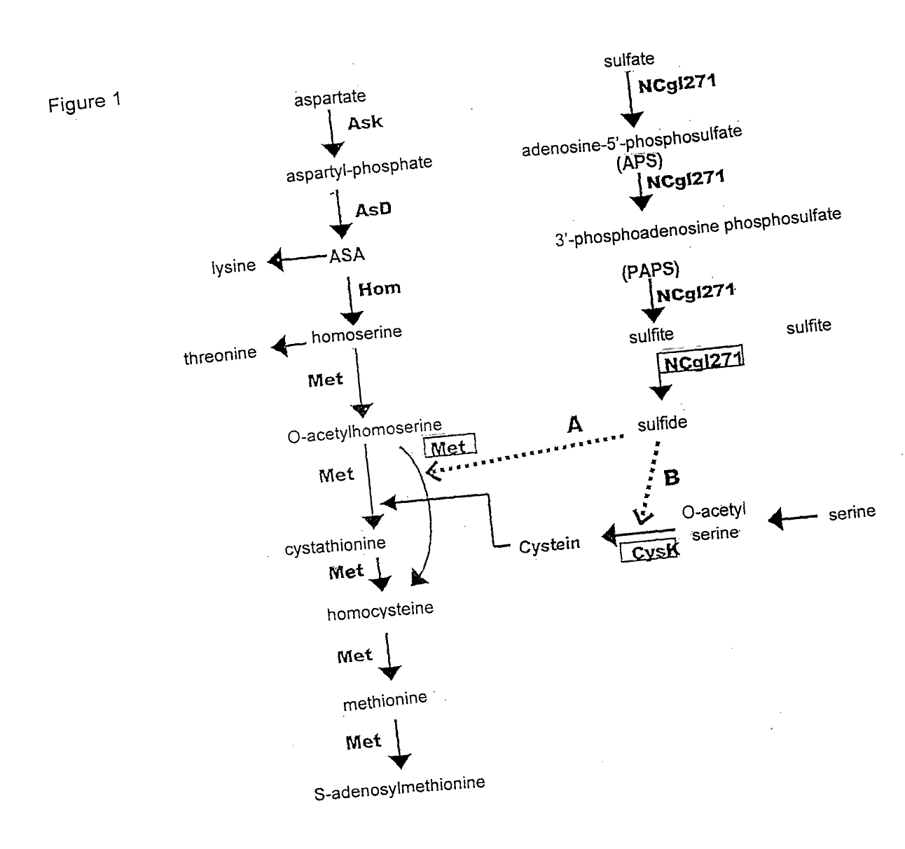

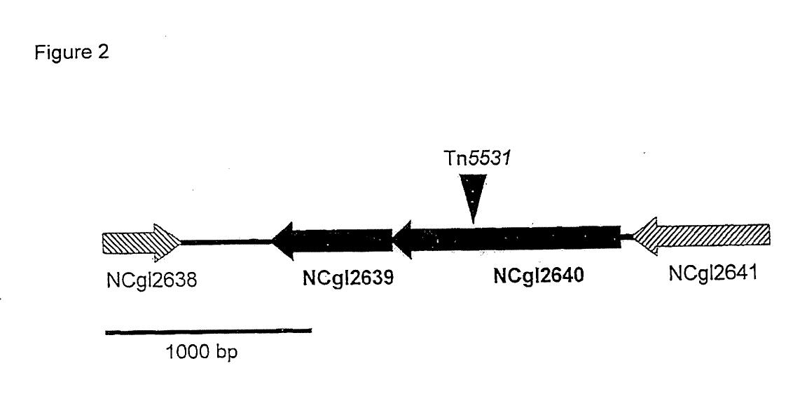

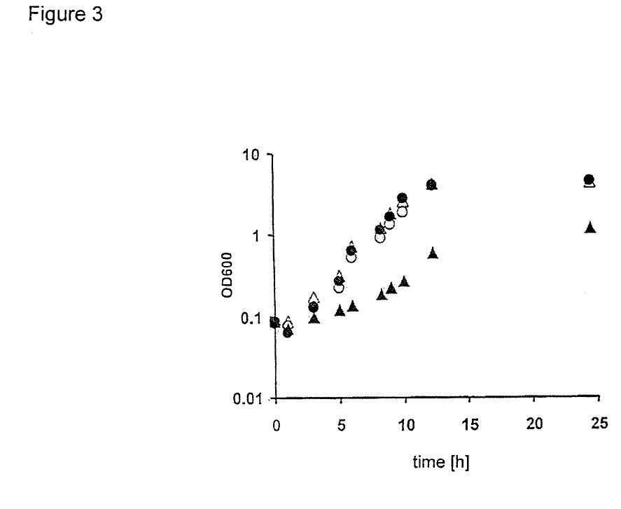

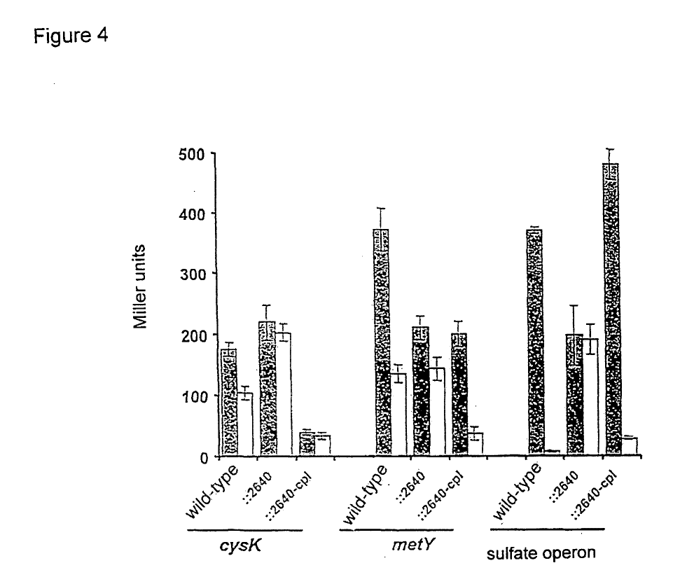

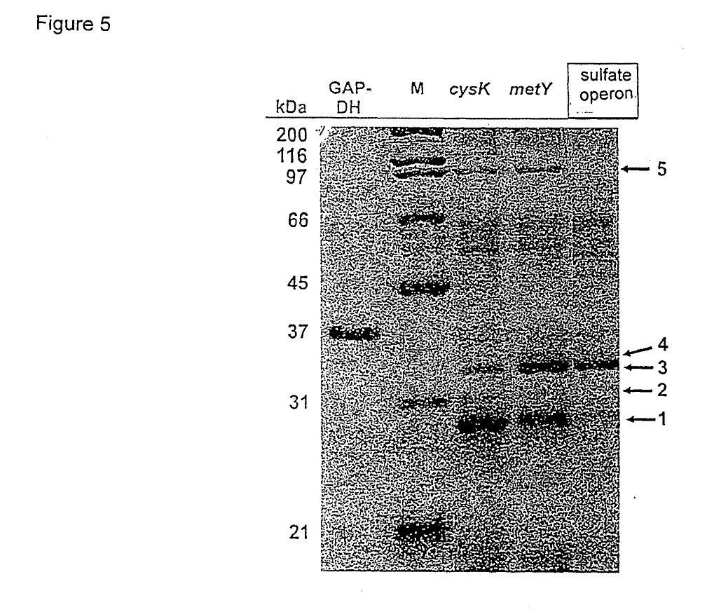

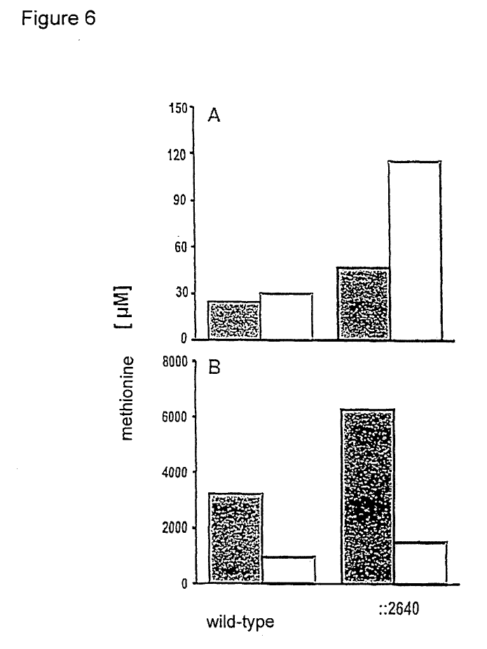

Sulfur can be incorporated either by direct sulhydrolation, which is catalyzed by MetY (A), or via the trans-sulfhydrolation pathway (B). lacZ fusions were prepared for promoters of the labeled gene products. The promoter of NCgl2718 regulates the expression of genes organized in the sulfate cluster (NCgl2715-NCgl2720).

The following abbreviations were used: Ask, aspartate kinase, AsDH, aspartate semialdehyde dehydrogenase, Hom, homoserine dehydrogenase, MetA, homoserine acetyltransferase, MetB, cystathionine-γ-synthase, MetC, cystathionine-β-lyase, MetH, methionine synthase, MetY, O-acetylhomoserine sulfhydrolase, MetK, S-adenosyl methionine synthase, NCgl2715, sulfate adenosyltransferase subunit 1; NCgl2716, sulfate adenosyltransferase subunit 2; NCgl2717, PAPS reductase; NCgl2718, sulfide reductase, which is annotated as putative nitrite reductase; CysK, cysteine synthase.

FIG. 2 The genomic context of NCgl2640

The GenBank annotations available for the ORFs:

NCgl2638, similarities to a multipartite Na⁺/H⁺ antiporter;
NCgl2639, similar to predicted hydrolases or acetyltransferases, alpha-/beta-hydrolase superfamily;
NCgl2640, hypothetic protein (uncharacterized BCR);
NCgl2641, hypothetic protein, no annotation;
Tn5531, transposon 5531 (IS1207). The filled triangle shows the insertion site of Tn5531 in NCgl2640.

FIG. 3 Growth curves of C. glutamicum wild-type and mutants cultivated in CGXII medium in the presence or absence of ethionine

Glucose concentration was 3 g l⁻¹; D,L-ethionine concentrations were 3.5 g l⁻¹ or 7.5 g l⁻¹ for the wild-type or mutant strain. It has to be observed that the wild-type does not grow in the presence of 7.5 g l⁻¹ D,L-ethionine.

Filled symbols: ethionine present during cultivation;
Open symbols: cultivation without ethionine;
▴ wild-type;
mutant strain.

FIG. 4 Influence of the NCgl2640 knockout on the methionine-dependent expression of cysK, metY and the sulfate operon

C. glutamicum-ATCC 14752 (wild-type; WT), the mutated strain 14752::2640, and mutated strains complemented with the plasmid-borne NCgl2640 (::2640-cp1) and containing the reporter plasmids pClik-PcysK, pClik-PmetY, or pClik-Psulfat were cultivated in CGXII (3 g/l glucose) medium in the presence or absence of methionine (10 mM). Promoter activity was determined by LacZ activity reporter assays and quantified in Miller units.

Dark bars: no methionine in the growth medium;
Empty bars: L-methionine in the growth medium.

FIG. 5 SDS-PAGE for proteins binding to putative promoter regions of the C. glutamicum genes for cysteine synthase (cysK), O-acetylhomoserine-sulfhydrolase (metY) and genes of the putative sulfate operon

1, regulator McbR, which is similar to TetR and was known from Rey et al. (vide supra);
2, exopoly phosphatase (degradation product);
3, exopoly phosphatase;
4, ATP phosphoribosyl transferase;
5, DNA polymerase I.

With the exception of the exopoly phosphatase and the DNA polymerase I, none of the proteins identified bound to the groES control promoter fragment.

GAP-DH, glyceraldehyde-3-phosphate-dehydrogenase was used as marker protein (37 kDa);

M, protein marker.

FIG. 6 Extra—(A) and intracellular (B) amounts of L-methionine in C. glutamicum wild-type and in the NCgl2640 knockout mutant strain (::2640) in the presence or absence of ethionine

Dark bars: no ethionine during incubation,
White bars: 3.5 g/l (wild-type) or 7.5 g/l (::2640) D,L-ethionine.

TABLE 1
Summary of the previously mentioned strains and plasmids.

| Strain or plasmid | Phenotype | Source or reference |
|---|---|---|
| C. glutamicum strains | | |
| ATCC 13032 | wild-type | ATCC |
| ATCC 14752 | wild-type | (Simic et al., 2001) |
| 13032::2640 | wild-type with NCgl2640, Km^{r} | according to the |
| | | present invention |
| 14752::2640 | wild-type with NCgl2640, Km^{r} | according to the |
| | | present invention |
| 14752-PcysK | ATCC 14752 with reporter | according to the |
| | plasmid pClik-PcysK, Cm^{r} | present invention |
| CG::2640-PcysK | 14752::2640 with reporter plasmid | according to the |
| | pClik-PcysK, Km^{r} Cm^{r} | present invention |
| CG::2640-PcysK-cpl | 14752::2640 with reporter plasmid | according to the |
| | pClik-PcysK-cpl, Km^{r} Cm^{r} | present invention |
| 14752-PmetY | ATCC 14752 with reporter | according to the |
| | plasmid pClik-PmetY, Cm^{r} | present invention |
| CG::2640-PmetY | 14752::2640 with reporter plasmid | according to the |
| | pClik-PmetY, Km^{r} Cm^{r} | present invention |
| CG::2640-PmetY-cpl | 14752::2640 with reporter plasmid | according to the |
| | pClik-PmetY-cpl, Km^{r} Cm^{r} | present invention |
| 14752-Psulfate | ATCC 14752 with reporter | according to the |
| | plasmid pClik-Psulfate, Cm^{r} | present invention |
| CG::2640-Psulfate | 14752::2640 with reporter plasmid | according to the |
| | pClik-Psulfate, Km^{r} Cm^{r} | present invention |
| CG::2640-Psulfate-cpl | 14752::2640 with reporter plasmid | according to the |
| | pClik-Psulfate-cpl, Km^{r} Cm^{r} | present invention |
| E. coli-strains | | |
| DH5α | F⁻ endA1, hsdR17(rk⁻mk⁺) supE44, | (Hanahan, 1983) |
| | thi-l λ⁻ recAI gyrA96 relA1 | |
| | Φ80ΔlacAm15 | |
| ET12567 | dam dcm hsd, restriction deficient | (MacNeil et al., |
| | | 1992) |
| Plasmids | | |
| pUC18 | Ap^{r}, lacZ | Stratagene |
| pCGL0040 | donor of Tn5531 (IS1207 Km^{r}); | (Ankri et al., 1996b) |
| | Ap^{r}, oriV_{E.c}. | |
| pMT1 | E. coli - C. glutamicum shuttle | (Follettie et al., 1993; |
| | vector, Ap^{r} Km^{r} | Lee and Sinskey, |
| | | 1994) |
| opClik-SacB | vector for allelic substitution by | (Hwang et al., 1999) |
| | homologous recombination; non- | |
| | replicative in C. glutamicum Km^{r}, | |
| | SacB | |
| pSdel-NCgl2640 | pClikSacB-based allelic | according to the |
| | substitution vector for | present invention |
| | chromosomal deletion of | |
| | NCgl2640 | |
| pClik-PcysK | cysK promoter probe vector lacZ | according to the |
| | fusion, Cm^{r} | present invention |
| pClik-PcysK-cpl | pClik-PcysK with NCgl2640, Cm^{r} | according to the |
| | | present invention |
| pClik-PmetY | metY promoter probe vector lacZ | according to the |
| | fusion, Cm^{r} | present invention |
| pClik-PmetY-cpl | pClik-PmetY with NCgl2640, Cm^{r} | according to the |
| | | present invention |
| pClik-Psulfate | Sulfate operon promoter probe | according to the |
| | vector lacZ fusion, Cm^{r} | present invention |
| pClik-Psulfate-cpl | pClik-Psulfate with NCgl2640, | according to the |
| | Cm^{r} | present invention |

### LITERATURE REFERENCES IN TABLE 1

Ankri S., Serebrijski, I., Reyes, O., and Leblon, G. (1996b) Mutations in the Corynebacterium glutamicum proline biosynthetic pathway: a natural bypass of the proA step. J Bacteriol 178, 4412-4419.
Follettie, M. T., Peoples, O. P., Agoropoulou, C., and Sinskey, A. J. (1993) Gene structure and expression of the Corynebacterium flavum N13 ask-asd operon. J Bacteriol 175, 4096-4103.
Hanahan, D. (1983) Studies on transformation of Escherichia coli with plasmids. J Mol Biol 166, 557-580.
Hwang, B.-J., Kim, Y., Kim, H.-B., Hwang, H.-J., Kim, J.-H., and Lee, H. S. (1999) Analysis of Corynebacterium glutamicum methionine biosynthetic pathway: isolation and analysis of metB encoding cystathionine γ-synthase. Mol Cells 9, 300-308.
Lee, H.-S., and Sinskey, A. J. (1994) Molecular characterization of AceB, a gene encoding malate synthase in Corynebacterium glutamicum. J Microbiol Biotechnol 4, 256-263.
MacNeil, D. J., Occi, J. L., Gewain, K. M., MacNeil, T., Gibbons, P. H., Ruby, C. L., and Danis, S. J. (1992) Complex organization of the Streptomyces avermitilis genes encoding the avermectin polyketide synthase. Gene 115, 119-125.
Simic, P., Sahm, H., and Eggeling, L. (2001) L-threonine export: use of peptides to identify a new translocator from Corynebacterium glutamicum. J Bacteriol 183, 5317-5324.

1. A microorganism for the production of a sulfur-containing compound, wherein the content and/or activity of at least one protein encoded by a nucleic acid molecule that is identical or functionally homologous to a nucleic molecule comprising SEQ ID NO. 1 is decreased as compared to the content and/or activity of said protein in a wild-type microorganism.

2. The microorganism according to claim 1, wherein the sulfur-containing compound is selected from the group consisting of: L-methionine, L-cysteine, L-homocysteine, L-cystathionine, S-adenosyl-L-methionine, glutathione, biotin, thiamine and/or lipoic acid, preferably L-methionine and/or L-cysteine.

3. The microorganism according to claim 1, wherein the microorganism is selected from the group consisting of: Actinobacteria, Cyanobacteria, Proteobacteria and/or Chloroflexus aurantiacus, preferably Corynebacteria, Mycobacteria, Streptomycetes, Salmonellae, Escherichia coli, Shigella, Bacillus, Serratia, Salmonella and/or Pseudomonas.

4. The microorganism according to claim 1, wherein said nucleic acid molecule is at least 30%, 40% or 50%, preferably to at least 60%, also preferably to at least 70%, especially preferably to at least 80%, particularly preferably to at least 90% and most preferably to at least 95% identical to SEQ ID NO:1.

5. The microorganism according to claim 1, wherein, the content and/or activity of at least one protein of the biosynthetic pathway of a sulfur-containing compound is increased and/or at least one nucleic acid molecule coding for a protein of the biosynthetic pathway of a sulfur-containing compound is mutated in such a way that the protein encoded by the nucleic acid molecule is not influenced in its activity by biosynthetic metabolites as compared to a wild-type microorganism.

6. The microorganism according to claim 5, wherein the nucleic acid molecule coding for a protein of the biosynthetic pathway of a sulfur-containing compound is selected from the group consisting of:
the nucleic acid coding for methionine synthase meth,
the nucleic acid coding for aspartate kinase lysC,
the nucleic acid coding for glycerinaldehyde-3-phosphate dehydrogenase gap,
the nucleic acid coding for 3-phosphoglycerate kinase pgk,
the nucleic acid coding for pyuvate carboxylase pyc,
the nucleic acid coding for triosephosphate isomerase tpi,
the nucleic acid coding for homoserine-O-acetyltransferase metA,
the nucleic acid coding for cystathionine-gamma-synthase metB,
the nucleic acid coding for cystathionine-gamma-lyase metC,
the nucleic acid coding for serine hydroxymethyltransferase glyA,
the nucleic acid coding for O-acetylhomoserine sulfhydrylase metY,
the nucleic acid coding for phosphoserine aminotransferase serC,
the nucleic acid coding for phosphoserine phosphatase serB,
the nucleic acid coding for serine acetyltransferase cysE,
the nucleic acid coding for homoserine dehydrogenase hom,
the nucleic acid coding for methionine synthase metE,
a nucleic acid coding for cysteine synthase,
a nucleic acid coding for sulfite reductase,
the nucleic acid coding for phosphoadenosine phosphosulfate reductase cysH,
a nucleic acid coding for sulfate adenylyltransferase subunit 1,
a nucleic acid coding for CysN sulfate adenylyltransferase subunit 2,
a nucleic acid coding for ferredoxin NADP reductase,
a nucleic acid coding for ferredoxin,
a nucleic acid coding for glucose-6-phosphate dehydrogenase, and
a nucleic acid coding for fructose-1,6-bisphosphatase.

7. The microorganism according to claim 6, wherein the nucleic acid molecule coding for a protein of the biosynthetic pathway of a sulfur-containing compound is functionally homologous to said nucleic acid molecule or preferably to at least 50%, preferably to at least 60%, also preferably to at least 70%, especially preferably to at least 80%, particularly preferably to at least 90% and most preferably to at least 95% identical to said nucleic acid molecule.

8. The microorganism according to claim 1 wherein, the content and/or activity of at least one protein of the biosynthetic pathway of a sulfur-containing compound is also decreased as compared to a wild-type microorganism.

9. The microorganism according to claim 8, wherein, a nucleic acid molecule coding for a protein of the biosynthetic pathway of a sulfur-containing compound is selected from the group consisting of:
the nucleic acid coding for homoserine kinase thrB,
the nucleic acid coding for threonine dehydratase ilvA,
the nucleic acid coding for threonine synthase thrC,
the nucleic acid coding for meso-diaminopimelate-D-dehydrogenase ddh,
the nucleic acid coding for phosphoenolpyruvate carboxykinase pck,
the nucleic acid coding for glucose-6-phosphate-6-isomerase pgi,
the nucleic acid coding for pyruvate oxidase poxB,
the nucleic acid coding for dihydrodipicolinate synthase dapA,
the nucleic acid coding for dihydrodipicolinate reductase dapB,
the nucleic acid coding for diaminopicolinate decarboxylase lysA,
a nucleic acid coding for glycosyltransferase, and/or
a nucleic acid coding for lactate dehydrogenase,

10. The microorganism according to claim 9, wherein, the nucleic acid molecule coding for a protein of the biosynthetic pathway of a sulfur-containing compound is functionally homologous to said nucleic acid molecule or preferably to at least 50%, preferably to at least 60%, also preferably to at least 70%, especially preferably to at least 80%, particularly preferably to at least 90% and most preferably to at least 95% identical to said nucleic acid molecule.

11. A method for the production of a sulfur-containing compound in microorganisms, comprising the cultivation of a microorganism according to claim 1.

12. The method according to claim 11, wherein the cultivation is a fermentation process, and wherein the sulfur-containing compounds are enriched in the medium and/or in the cells of the microorganisms and are isolated there from.

13. The method according to claim 11, wherein the sulfur-containing compound is selected from the group consisting of: L-methionine, L-cysteine, L-homocysteine, L-cystathionine, S-adenosyl-L-methionine, glutathione, biotin, thiamine and/or lipoic acid, preferably L-methionine and/or L-cysteine.

14. The method according to claim 11, wherein the microorganism is selected from the group consisting of: Actinobacteria, Cyanobacteria, Proteobacteria, Chloroflexus aurantiacus, Pirellula sp. 1, Halobacteria and/or Methanococci, preferably Corynebacteria, Mycobacteria, Streptomycetes, Salmonellae, Escherichia coli, Shigella and/or Pseudomonas.

15. The method according to claim 14, wherein the microorganism is Corynebacterium glutamicum.

16. The method according to claim 11, wherein the content and/or activity of proteins, which are encoded by a nucleic acid molecule that is identical or functionally homologous to a nucleic acid molecule having the sequence of SEQ ID NO: 1 is decreased by disruption and/or deletion of the corresponding genomic nucleic acid sequence(s).

17. The method according to claim 16, wherein the content and/or activity of proteins, which are encoded by a nucleic acid molecule that is identical or functionally homologous to a nucleic acid molecule having the sequence of SEQ ID NO: 1 is decreased by disruption and/or the content and/or activity of proteins, which are encoded by a nucleic acid molecule that is identical or functionally homologous to a nucleic acid molecule having the sequence of SEQ ID NO: 1 are decreased by the following steps:
a) Producing a vector, comprising the following nucleic acid sequences in 5′ to 3′ orientation:
a promoter sequence functional in microorganisms,
operatively linked thereto a DNA sequence that is identical or functionally homologous to the 5′ end of the sequence SEQ ID NO: 1,
operatively linked thereto a DNA sequence coding for a resistance gene,
operatively linked thereto a DNA sequence that is identical or functionally homologous to the 3′ end of the sequence SEQ ID NO: 1,
operatively linked thereto a termination sequence functional in microorganisms, and
b) transferring the vector from a) to the microorganism and, optionally, integrating the vector into its genome.

18. The method according to claim 11, wherein the content and/or activity of proteins, which are encoded by nucleic acids that are identical or functionally homologous to nucleic acids having the sequence of SEQ ID NO: 1 are decreased by the following steps:
a) producing a vector, comprising the following nucleic acid sequences in 5′ to 3′ orientation:
a promoter sequence functional in the respective microorganism,
operatively linked thereto an antisense sequence to the sequence of SEQ ID NO: 1 or a functional homolog thereof,
operatively linked thereto a termination sequence functional in the microorganism, and
b) transferring the vector from a) to the microorganism and, optionally, integrating the vector into its genome.

19. The method according to claim 11, wherein the content and/or activity of proteins, which are encoded by a nucleic acid molecule that is identical or functionally homologous to a nucleic acid molecule having the sequence of SEQ ID NO: 1 are decreased by the following steps:
a) producing a vector, comprising the following nucleic acid sequences in 5′ to 3′ orientation:
a promoter sequence functional in the respective microorganism,
operatively linked thereto a nucleic acid sequence, which is complementary to the sequence identified in SEQ ID NO: 1 or a functional homolog thereof or parts thereof,
operatively linked thereto a DNA sequence coding for ribonuclease P,
operatively linked thereto a termination sequence functional in the respective microorganism, and
b) transferring the vector from a) to the microorganism and, optionally, integrating the vector into its genome.

20. The method according to claim 11, wherein the content and/or activity of proteins, which are encoded by a nucleic acid molecule that is identical or functionally homologous to a nucleic acid molecule having the sequence identified in SEQ ID NO: 1 are decreased by the following steps:
a) producing a vector, comprising the following nucleic acid sequences in 5′ to 3′ orientation:
a promoter sequence functional in the respective microorganism,
operatively linked thereto a nucleic acid sequence coding for a ribozyme, which specifically recognizes the mRNA of a nucleic acid having the sequence of SEQ ID NO: 1 or a functional homolog thereof,
operatively linked thereto a termination sequence functional in the respective microorganism, and
b) transferring the vector from a) to the microorganism and, optionally, integrating the vector into its genome.

21. The method according to claim 11, wherein the content and/or activity of proteins, which are encoded by a nucleic acid molecule that is identical or functionally homologous to a nucleic acid molecule having the sequence of SEQ ID NO: 1, are decreased by the expression of at least one recombinant antibody, which is specific for the proteins and blocks the function of the proteins in the metabolism of sulfur-containing compounds.

22. The method according to claim 11, wherein the content and/or activity of proteins, which are encoded by a nucleic acid molecule that is identical or functionally homologous to a nucleic acid molecule having the sequence of SEQ ID NO: 1, are decreased by the expression of at least one non-functional nucleic acid, which as compared to the nucleic acid molecule having the sequence of SEQ ID NO: 1 or its homologs or parts thereof contains point mutation(s), deletion(s) and/or insertion(s).

23. The method according to claim 11, wherein the functional expression of a nucleic acid molecule that is identical or functionally homologous to a nucleic acid molecule having the sequence of SEQ ID NO: 1 is essentially completely suppressed as compared to the wild-type of the microorganism.

24. The method according to claim 11, wherein additionally the content and/or activity of proteins, which are encoded by a nucleic acid molecule that is identical or functionally homologous to a nucleic acid molecule having the sequence of SEQ ID NO: 3, is decreased as compared to the wild-type of the microorganism.

25. The method according to claim 24, wherein the functional expression of a nucleic acid molecule that is identical or functionally homologous to a nucleic acid molecule having the sequence of SEQ ID NO: 3 are essentially completely suppressed as compared to the wild-type of the microorganism.

26. The method according to claim 11, wherein additionally the functional expression of at least one nucleic acid according to claim 6 or 7 is increased and/or the functional expression of at least one nucleic acid according to claim 9 or 10 is suppressed.

27. Use of a microorganism according to claim 1 for the production of a sulfur-containing compound, especially for the production of L-methionine and/or L-cysteine.

28. Use of a nucleic acid molecule that is identical or functionally homologous to a nucleic acid molecule having the sequence of SEQ ID NO: 1 for the production of a sulfur-containing compound, especially for the production of L-methionine and/or L-cysteine.
